(19) United States
(12) Patent Application Publication
(10) Pub. No.: US 2025/0340865 A1
(43) Pub. Date: Nov. 6, 2025

(54) GENE VARIANT LIBRARIES AND METHODS OF USE THEREOF

(71) Applicant: Watchmaker Genomics, Inc., Boulder, CO (US)

(72) Inventors: Leonidas Karamanof, Cape Town (ZA); Eric van der Walt, Cape Town (ZA); Bjarne Faurholm, Cape Town (ZA); Michiel Theodoor van Diepen, Cape Town (ZA); David Lloyd Matten, Cape Town (ZA); Campbell McDuling, Cape Town (ZA); Daniel Henri Le Jeune, Cape Town (ZA); Thomas Harrison, Boulder, CO (US); Brian Kudlow, Boulder, CO (US)

(73) Assignee: Watchmaker Genomics, Inc., Boulder, CO (US)

(21) Appl. No.: 19/098,542

(22) Filed: Apr. 2, 2025

Related U.S. Application Data
(60) Provisional application No. 63/573,241, filed on Apr. 2, 2024.

(51) Int. Cl. C12N 15/10; C12Q 1/48; C12Q 1/6806; C12Q 1/6869; C40B 40/08

(52) U.S. Cl. CPC C12N 15/1072; C12Q 1/485; C12Q 1/6806; C12Q 1/6869; C40B 40/08; G01N 2333/91245

(57) ABSTRACT

Disclosed herein, in part, are methods and compositions for performing high throughput screens of enzyme activity.

### RELATED APPLICATIONS

This application claims benefit under 35 U.S.C. § 119(e) to U.S. Provisional Application No. 63/573,241, filed Apr. 2, 2024, entitled “Gene Variant Libraries and Methods and Use Thereof”, the entire contents of which is incorporated herein by reference.

### REFERENCE TO AN ELECTRONIC SEQUENCE LISTING

The contents of the electronic sequence listing (W109470005US01-SEQ-EMB.xml; Size: 59,172 bytes; and Date of Creation: Jul. 3, 2025) are herein incorporated by reference in its entirety.

### BACKGROUND

A central challenge in protein engineering is testing the activities of hundreds to tens of thousands of possible protein variants to determine which proteins have improved properties for a given application.

### SUMMARY

This disclosure provides compositions and methods for measuring the relative activity of different variant gene products of a given gene. Previous methods for making these types of measurements combined high throughput gene synthesis and a measurement of variant gene product activity (e.g., sequencing) to measure the activity of many different variant gene products. Many assays work by linking the activity of the variant gene product to the amount of DNA, RNA or protein that corresponds to the variant gene product, so that a measurement of the amount of DNA, RNA or protein can be used to infer activity. Some of these assays separate the different variants into different partitions (e.g., using microfluidics), measure variant activity in each droplet, and from the droplets identify variants with improved activity. These assays are limited because they don't measure the activity of every variant and thus do not directly identify intermediate and low activity variants, which may be useful in some applications or in downstream gene product design. These assays could use next-generation sequencing to measure production of DNA or RNA by the gene products, but these measurements could be biased by errors in sample preparation and sequencing (e.g., next-generation sequencing). This is particularly problematic when two or more polynucleotides that encode different variants of the same gene product only differ by one nucleotide (i.e., having a Hamming distance of one). In this case, a sequencing error at the position of the one nucleotide results in an incorrect mapping between the variant gene product and its function. Compositions and methods provided herein present solutions to these sequencing limitations in high throughput methods for determining the activity of hundreds to tens of thousands of different variants of the same gene product. The plurality of polynucleotides encoding these protein variants is comprised in a library.

The inventors discovered that a synonymous codon (e.g., a silent mutation that doesn't change the amino acid sequence of the variant) could be introduced into the DNA encoding the gene product to increase the Hamming distance between DNA variants that only differ by a Hamming distance of one. With a Hamming distance of two or greater between variants, the odds of sequencing error go down tremendously, which results in a more accurate and precise assay. In some cases, more than one synonymous codon can be introduced into a given DNA variant encoding a protein variant to make the Hamming distance from that DNA variant to every other DNA variant at that position greater than 2.

The inventors of the instant application exemplified this library design strategy by combining it with compartmentalized self-replication (CSR) and shotgun sequencing to measure the fitness of thousands of different variant gene products relative to a control. Results show that increasing Hamming distance to greater than 1 using synonymous codons greatly increases assay accuracy and precision.

In some aspects, this disclosure provides a library comprising a plurality of polynucleotides that encode different variants of the same gene product, wherein the plurality comprises: (i) a first polynucleotide comprising nucleic acids encoding a first variant, the nucleic acids comprising a first non-synonymous codon at a first position and a synonymous codon (e.g., a first synonymous codon); and (ii) a second polynucleotide comprising nucleic acids encoding a second variant, the nucleic acids comprising a second non-synonymous codon at the first position and a synonymous codon (e.g., a second synonymous codon).

In some aspects, this disclosure provides a library comprising a plurality of polynucleotides that encode different variants of the same gene product, wherein the plurality comprises: (i) a first polynucleotide comprising nucleic acids encoding a first variant, the nucleic acids comprising a first non-synonymous codon at a first position and a first synonymous codon; and (ii) a second polynucleotide comprising nucleic acids encoding a second variant, the nucleic acids comprising a second non-synonymous codon at the first position and a second synonymous codon, wherein the first non-synonymous codon is different than the second non-synonymous codon.

In some embodiments, the plurality of polynucleotides comprises a third polynucleotide, wherein the third polynucleotide comprises nucleic acids encoding a third variant, the nucleic acids comprising: (i) a non-synonymous codon at the first position that is different from the first non-synonymous codon and the second non-synonymous codon; and (ii) a synonymous codon (e.g., a third synonymous codon).

In some embodiments, the plurality of polynucleotides comprises a fourth polynucleotide, wherein the fourth polynucleotide comprises: nucleic acids encoding a fourth variant, the nucleic acids comprising: (i) a non-synonymous codon at a different position than the first position; and (ii) a synonymous codon (e.g., the fourth synonymous codon).

In some embodiments, the first position is within 200 nucleotides of the synonymous codon. In some embodiments, the first position is within 30 nucleotides of the synonymous codon. In some embodiments, the first position is within 3 nucleotides of the synonymous codon.

In some embodiments, the nucleic acids of the first polynucleotide, the nucleic acids of the second polynucleotide, the nucleic acids of the third polynucleotide, and/or the nucleic acids of the fourth polynucleotide further comprise an additional synonymous codon.

In some embodiments, the first polynucleotide comprises, in consecutive codons: a synonymous codon; the first non-synonymous codon; and an additional synonymous codon. In some embodiments, the second polynucleotide comprises in consecutive codons: a synonymous codon; the second non-synonymous codon; and an additional synonymous codon. In some embodiments, the third polynucleotide comprises, in consecutive codons: a synonymous codon; the non-synonymous codon at the first position that is different from the first non-synonymous codon and the second non-synonymous codon; and an additional synonymous codon.

In some embodiments, the fourth polynucleotide comprises, in consecutive codons: a synonymous codon; the non-synonymous codon at a different position than the first position; and an additional synonymous codon. In some embodiments, the first polynucleotide and the second polynucleotide differ by a Hamming distance of at least 2.

In some embodiments, the first non-synonymous codon encodes a specific amino acid and is selected to have a codon usage percentage that is closest to the codon usage percentage of a codon at the first position of a polynucleotide that encodes a reference sequence of the gene product; the second non-synonymous codon encodes a specific amino acid and is selected to have a codon usage percentage that is closest to a codon usage percentage of a codon at the second position of a polynucleotide that encodes a reference sequence of the gene product; the third non-synonymous codon encodes a specific amino acid and is selected to have a codon usage percentage that is closest to a codon usage percentage of a codon at the third position of a polynucleotide that encodes a reference sequence of the gene product; and/or the fourth non-synonymous codon encodes a specific amino acid and is selected to have a codon usage percentage that is closest to a codon usage percentage of a codon at the fourth position of a polynucleotide that encodes a reference sequence of the gene product.

In some embodiments, the reference sequence encodes the wild-type amino acid sequence of the gene product.

In some embodiments, the reference sequence comprises the wild-type nucleic acid sequence of the gene product.

In some embodiments, the first non-synonymous codon has a codon usage percentage that is within 10% of the codon usage percentage of a codon at the first position of a polynucleotide that encodes a reference sequence of the gene product; the second non-synonymous codon has a codon usage percentage that is within 10% of the codon usage percentage of a codon at the second position of a polynucleotide that encodes a reference sequence of the gene product; the third non-synonymous codon has a codon usage percentage that is within 10% of the codon usage percentage of a codon at the third position of a polynucleotide that encodes a reference sequence of the gene product; and/or the fourth non-synonymous codon has a codon usage percentage that is within 10% of the codon usage percentage of a codon at fourth position of a polynucleotide that encodes a reference sequence of the gene product.

In some embodiments, the library further comprises additional polynucleotides that each comprise nucleic acids encoding different variants of the gene product, wherein the nucleic acids of each of the additional polynucleotides comprise: (i) a non-synonymous codon; and (ii) a synonymous codon.

In some embodiments, the non-synonymous codon is within 200 nucleotides of the synonymous codon. In some embodiments, the non-synonymous codon is within 30 nucleotides of the synonymous codon. In some embodiments, the non-synonymous codon is within 3 nucleotides of the synonymous codon. In some embodiments, the nucleic acids of at least one of the additional polynucleotides further comprises an additional synonymous codon.

In some embodiments, at least one of the additional polynucleotides comprises, in consecutive codons: a synonymous codon; the non-synonymous codon; and an additional synonymous codon.

In some embodiments, 1%-100% of the first polynucleotide, the second polynucleotide, the third polynucleotide, the fourth polynucleotide, and the additional polynucleotides differ by a Hamming distance of at least 2.

In some embodiments, the first polynucleotide, the second polynucleotide, the third polynucleotide, and the additional polynucleotides collectively comprise nucleic acids encoding at least 75% of possible amino acid substitutions at the first position.

In some embodiments, the first polynucleotide, the second polynucleotide, the third polynucleotide, and the additional polynucleotides collectively comprise nucleic acids encoding at least 90% of possible amino acid substitutions at the first position.

In some embodiments, the first polynucleotide, the second polynucleotide, the third polynucleotide, and the additional polynucleotides collectively comprise nucleic acids encoding 100% of possible amino acid substitutions at the first position.

In some embodiments, the first polynucleotide, the second polynucleotide, the third polynucleotide, the fourth polynucleotide, and the additional polynucleotides collectively comprise nucleic acids encoding at least 10% of possible single amino acid substitutions of the gene product. In some embodiments, the first polynucleotide, the second polynucleotide, the third polynucleotide, the fourth polynucleotide, and the additional polynucleotides collectively comprise nucleic acids encoding at least 50% of possible single amino acid substitutions of the gene product. In some embodiments, the first polynucleotide, the second polynucleotide, the third polynucleotide, the fourth polynucleotide, and the additional polynucleotides collectively comprise nucleic acids encoding at least 75% of possible single amino acid substitutions of the gene product.

In some embodiments, the first polynucleotide, the second polynucleotide, the third polynucleotide, the fourth polynucleotide, and the additional polynucleotides collectively comprise nucleic acids encoding at least 90% of possible single amino acid substitutions of the gene product. In some embodiments, the first polynucleotide, the second polynucleotide, the third polynucleotide, the fourth polynucleotide, and the additional polynucleotides collectively comprise nucleic acids encoding at least 95% of possible single amino acid substitutions of the gene product. In some embodiments, the first polynucleotide, the second polynucleotide, the third polynucleotide, the fourth polynucleotide, and the additional polynucleotides collectively comprise nucleic acids encoding at least 99% of possible single amino acid substitutions of the gene product.

In some embodiments, the gene product comprises a nucleic acid modifying enzyme. In some embodiments, the nucleic acid modifying enzyme is a DNA polymerase. In some embodiments, the nucleic acid modifying enzyme is a Family A polymerase. In some embodiments, the nucleic acid modifying enzyme is a Family B polymerase.

In some embodiments, the DNA polymerase is a Thermococcus kodakarensis (KOD) DNA polymerase or a Taq polymerase. In some embodiments, the first polynucleotide, the second polynucleotide, the third polynucleotide, the fourth polynucleotide, and the additional polynucleotides collectively comprise nucleic acids encoding at least 25% of the possible single non-synonymous codons in the gene product. In some embodiments, the first polynucleotide, the second polynucleotide, the third polynucleotide, the fourth polynucleotide, and the additional polynucleotides collectively comprise nucleic acids encoding at least 50% of the possible single non-synonymous codons of the gene product. In some embodiments, the first polynucleotide, the second polynucleotide, the third polynucleotide, the fourth polynucleotide, and the additional polynucleotides collectively comprise nucleic acids encoding at least 75% of the possible single non-synonymous codons of the gene product. In some embodiments, the first polynucleotide, the second polynucleotide, the third polynucleotide, the fourth polynucleotide, and the additional polynucleotides collectively comprise nucleic acids encoding at least 95% of the possible single non-synonymous codon of the gene product.

In some embodiments, the plurality of polynucleotides comprises a polynucleotide comprising nucleic acids encoding a wild-type of the gene product. In some embodiments, the first polynucleotide, the second polynucleotide, the third polynucleotide, the fourth polynucleotide, and the additional polynucleotides collectively comprise nucleic acids encoding at least 100 different variants of the same gene product. In some embodiments, the first polynucleotide, the second polynucleotide, the third polynucleotide, the fourth polynucleotide, and the additional polynucleotides collectively comprise nucleic acids encoding at least 500 different variants of the same gene product.

In some embodiments, the first polynucleotide, the second polynucleotide, the third polynucleotide, the fourth polynucleotide, and the additional polynucleotides collectively comprise nucleic acids encoding at least 5,000 different variants of the same gene product. In some embodiments, the first polynucleotide, the second polynucleotide, the third polynucleotide, the fourth polynucleotide, and the additional polynucleotides collectively comprise nucleic acids encoding at least 10,000 different variants of the same gene product. In some embodiments, the first polynucleotide, the second polynucleotide, the third polynucleotide, the fourth polynucleotide, and the additional polynucleotides collectively comprise nucleic acids encoding at least 20,000 different variants of the same gene product. In some embodiments, the first polynucleotide, the second polynucleotide, the third polynucleotide, the fourth polynucleotide, and the additional polynucleotides collectively comprise nucleic acids encoding at least 30,000 different variants of the same gene product.

In some embodiments, each polynucleotide encodes a single gene variant of the same gene product.

In some embodiments, the nucleic acids of the first polynucleotide, the nucleic acids of the second polynucleotide, the nucleic acids of the third polynucleotide, the nucleic acids of the fourth polynucleotide, and/or the nucleic acids of an additional polynucleotide comprise a synonymous codon or non-synonymous codon that introduces a restriction site. In some embodiments, the nucleic acids of the first polynucleotide, the nucleic acids of the second polynucleotide, the nucleic acids of the third polynucleotide, the nucleic acids of the fourth polynucleotide, and/or the nucleic acids of an additional polynucleotide comprise a synonymous codon that introduces a restriction site.

In some embodiments, this disclosure provides a library comprising a plurality of polynucleotides that encode different variants of the same gene product, wherein the plurality comprises: (i) a first polynucleotide comprising nucleic acids encoding a first variant, wherein the first polynucleotide comprises a first non-synonymous codon and a synonymous codon; and (ii) a second polynucleotide comprising nucleic acids encoding a second variant, wherein the second polynucleotide comprises a second non-synonymous codon and a synonymous codon.

In some embodiments, the plurality comprises a synonymous polynucleotide comprising nucleic acids of a gene variant encoding the gene product, wherein the synonymous polynucleotide comprises a first synonymous codon and a second synonymous codon.

In some embodiments, this disclosure provides a library comprising a plurality of polynucleotides that encode different variants of the same gene, wherein the plurality comprises a synonymous polynucleotide comprising nucleic acids of a gene variant encoding the gene product, wherein the synonymous polynucleotide comprises a first synonymous codon and a second synonymous codon.

In some embodiments, the first synonymous codon is within 200 nucleotides of the second synonymous codon on the first polynucleotide. In some embodiments, the first synonymous codon is within 30 nucleotides of the second synonymous codon on the first polynucleotide.

In some embodiments, the first synonymous codon is within 3 nucleotides of the second synonymous codon on the first polynucleotide. In some embodiments, the synonymous polynucleotide comprises a third synonymous codon. In some embodiments, the synonymous polynucleotide comprises, in consecutive codons: the first synonymous codon; the second synonymous codon; and the third synonymous codon. In some embodiments, this disclosure provides a library comprising the plurality of polynucleotides described herein.

In some embodiments, a polynucleotide of the plurality of polynucleotides comprises a barcode. In some embodiments, this disclosure provides a plurality of plasmids that comprise a polynucleotide of the library. In some embodiments, this disclosure provides a plurality of vectors that encode a polynucleotide of the library described herein. In some embodiments, this disclosure provides a plurality of cells that comprise: (i) a polynucleotide of the library; (ii) a plasmid of the plurality of plasmids; and/or (iii) a vector of the plurality of vectors.

In some embodiments, this disclosure provides methods of producing a plurality of polynucleotides, the methods comprising synthesizing the first polynucleotide and the second polynucleotide. In some embodiments, the method further comprises synthesizing the third polynucleotide. In some embodiments, the method further comprises synthesizing the fourth polynucleotide. In some embodiments, the method further comprises synthesizing at least one of the additional polynucleotides.

In some embodiments, the nucleic acids of the first polynucleotide, the nucleic acids of the second polynucleotide, the nucleic acids of the third polynucleotide, the nucleic acids of the fourth polynucleotide, the nucleic acids of the at least one of the additional polynucleotides, and/or the nucleic acids of the synonymous polynucleotide further comprise an additional nonsynonymous codon.

In some embodiments, this disclosure provides methods for determining the relative activity of a plurality of nucleic acid modifying enzyme variants of the same gene, the methods comprising: obtaining a plurality of cells, wherein at least one of the cells has been transformed with a polynucleotide of the plurality of polynucleotides; depositing, into different partitions, (i) individual cells of the plurality of cells, and (ii) reagents sufficient for an active nucleic acid modifying enzyme variant to produce an identifying nucleic acid product that can be used to identify the active nucleic acid modifying enzyme variant in the partition; lysing the individual cells in the different partitions to combine (a) nucleic acid modifying enzyme variants produced by the cell in the partition and (b) the reagents sufficient for an active nucleic acid modifying enzyme variant to produce an identifying nucleic acid product; performing sequencing to determine the amount of the identifying nucleic acid product in at least one of the different partitions; and determining the relative activity of a plurality of nucleic acid modifying enzyme variants by comparing the amount of at least one identifying nucleic acid product to an amount of identifying nucleic acid products produced in a partition comprising a control nucleic acid modifying enzyme variant.

In some embodiments, at least 2 cells of the plurality of cells have been transformed with a polynucleotide of the plurality of polynucleotides. In some embodiments, each cell of the plurality of cells has been transformed with a polynucleotide of the plurality of polynucleotides. In some embodiments, the control nucleic acid modifying enzyme variant comprises a wild-type nucleic acid modifying enzyme of the same gene as the nucleic acid modifying enzyme variants. In some embodiments, the control nucleic acid modifying enzyme variant comprises a nucleic acid modifying variant of known function.

In some embodiments, the control nucleic acid modifying enzyme variant comprises a synonymous codon.

In some embodiments, the different partitions are different wells in a plate or different vials. In some embodiments, the different partitions are water-in-oil emulsions. In some embodiments, the lysing the individual cells comprises lysing the individual cells with at least one surfactant and/or lysing individuals cells using heat. In some embodiments, determining the relative activity of a nucleic acid modifying enzyme variant comprises determining an enrichment of an identifying nucleic acid product corresponding to the nucleic acid modifying enzyme variant relative to the control nucleic acid modifying enzyme variant.

In some embodiments, performing sequencing comprises performing shotgun sequencing. In some embodiments, performing sequencing comprises performing long-read sequencing.

### BRIEF DESCRIPTION OF THE DRAWINGS

FIGS. 1A-1D relate to the design of libraries of polynucleotides encoding variants of an enzyme. FIG. 1A shows nucleotide changes (marked) and resulting amino acid mutations (marked). Each paired row of nucleotides and corresponding amino acids represents one variant. Variants comprise both synonymous codons (nucleotide changes that result in the same amino acid as the wild-type enzyme) and non-synonymous codons (nucleotide changes that result in different amino acids from the wild-type enzyme). FIG. 1B shows the nucleotide changes (marked) that result in the substitutions listed on the left. FIG. 1C shows the Hamming distance from the wild-type enzyme sequence (top) and the frequency of variants with a pairwise distance of 1, 2, 3, 4, 5, 6, 7, 8, and 9 base pairs. FIG. 1D shows the frequencies of pairwise Hamming distances between each member of the library and every other member of the library.

FIG. 2 shows a workflow schematic of CSR to characterize the performance and phenotype of Taq DNA polymerase variants.

FIG. 3 shows the amino acids at each position (x-axis) as a relative fold change compared to pre-CSR (y-axis), representing a comprehensive survey of the sequence-function relationship of all single substitutions.

FIGS. 4A-4B show the distributions of Hamming distances between variant sequences and the reference sequence in (FIG. 4A), an exemplary standard three-nucleotide codon substitution library design, versus (FIG. 4B), an exemplary supercodon substitution library design.

FIGS. 5A-5B show the distributions of sequence variant frequencies observed after sequencing (FIG. 5A), an exemplary standard three-nucleotide codon substitution library design, versus (FIG. 5B), an exemplary supercodon substitution library design.

### DETAILED DESCRIPTION

In some embodiments, this disclosure describes a library comprising a plurality of polynucleotides comprising nucleic acids that encode different variants of the same gene product, wherein the plurality comprises: (i) a first polynucleotide comprising nucleic acids encoding a first variant, the nucleic acids comprising a first non-synonymous codon or synonymous codon at a first position and a synonymous codon; and (ii) a second polynucleotide comprising nucleic acids encoding a second variant, the nucleic acids comprising a second non-synonymous codon or synonymous codon at the first position and a synonymous codon. Unless otherwise stated, the non-synonymous and synonymous codons described herein are located in nucleic acids encoding a gene product.

### Libraries

A “library” refers to a composition comprising a plurality of polynucleotides. In some embodiments, the library comprises a buffered solution sufficient for the storage of polynucleotides. In some embodiments, the library comprises lyophilized polynucleotides. In some embodiments, polynucleotides of the plurality of polynucleotides encode different variants of the same gene. In some embodiments, the library comprises other polynucleotides that are not polynucleotides of the plurality of polynucleotides. In some embodiments, a library comprises a plurality of polynucleotides described herein (e.g., first polynucleotides, second polynucleotides, third polynucleotides, fourth polynucleotides and/or additional polynucleotides).

A “polynucleotide” refers to a polymer of nucleotides. A polynucleotide is generally composed of nucleotides that are naturally found in DNA or RNA (e.g., adenosine/deoxyadenosine (A), thymidine/deoxythymidine (T), guanosine/deoxyguanosine (G), cytidine/deoxycytidine (C) and uridine (U) joined by phosphodiester bonds. Polynucleotides may also comprise nucleotides or nucleotide analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. A polynucleotide sequence presented herein is presented in a 5′ to 3′ direction unless otherwise indicated. In some embodiments, a polynucleotide encodes a variant gene product. The polynucleotide may also comprise additional nucleic acids that are not encoding the variant gene product. In some embodiments, the polynucleotide comprises a promoter or a terminator. In some embodiments, the polynucleotide comprises a barcode that is indicative of the variant gene product encoded by the polynucleotide. A “barcode” refers to a nucleic acid molecule (e.g., RNA or DNA) whose sequence is used to identify a given gene variant, or an individual nucleic acid molecule. For example, a polynucleotide may comprise, from 5′ to 3′ a barcode and then nucleic acids encoding a variant gene product. For example, a polynucleotide may comprise, from 5′ to 3′ nucleic acids encoding a variant gene product and then a barcode. In some embodiments, the barcode comprises at least 5 nucleotides. In some embodiments, the barcode comprises at least 6 nucleotides. In some embodiments, the barcode comprises at least 8 nucleotides. In some embodiments, the barcode comprises at least 10 nucleotides. In some embodiments, the barcode comprises at least 20 nucleotides. In some embodiments, the barcode is 6-20 nucleotides. In some embodiments, the barcode is randomly generated. In some embodiments, the polynucleotide comprises between about 100 and about 20,000 nucleotides. In some embodiments, the polynucleotide comprises between about 100 and about 10,000 nucleotides. In some embodiments, the polynucleotide comprises between about 100 and about 8.000 nucleotides. In some embodiments, the polynucleotide comprises between about 5000 and about 8.000 nucleotides. In some embodiments, the polynucleotide comprises between about 100 and about 3,000 nucleotides. In some embodiments, the polynucleotide comprises between about 100 and about 2,000 nucleotides. In some embodiments, the polynucleotide comprises at most 3,000 nucleotides.

A “plurality” refers to at least 2. In some embodiments, a plurality refers to at least 5, at least 10, at least 50, at least 100, at least 500, at least 1000, at least 2000, at least 5000, at least 10,000, at least 20,000, at least 50,000, at least 100,000, at least 250,000, at least 500,000, at least 750,000, at least 1,000,000, at least 5,000,000, or at least 10,000,000, or more. In some embodiments, a plurality refers to 2-20,000. In some embodiments, a plurality refers to 100-1000. In some embodiments, a plurality refers to 500-5,000. In some embodiments, a plurality refers to 1000-10,000.

A “plurality of polynucleotides” refers to at least 2 polynucleotides. For example, a plurality of polynucleotides may refer to at least 5 polynucleotides, at least 10 polynucleotides, at least 50 polynucleotides, at least 100 polynucleotides, at least 500 polynucleotides, at least 1,000 polynucleotides, at least 2,000 polynucleotides, at least 5,000 polynucleotides, at least 10,000 polynucleotides, at least 20,000 polynucleotides, at least 50,000 polynucleotides, at least 100,000 polynucleotides, at least 250,000 polynucleotides, at least 500,000 polynucleotides, at least 750,000 polynucleotides, at least 1,000,000 polynucleotides, at least 5,000,000 polynucleotides, or at least 10,000,000 polynucleotides. In some embodiments, a plurality of polynucleotides may refer to at least 108 polynucleotides, at least 1010 polynucleotides, at least 1012 polynucleotides, at least 1014 polynucleotides, at least 1016 polynucleotides, at least 1018 polynucleotides, at least 1020 polynucleotides, at least 1022 polynucleotides, or at least 1024 polynucleotides. In some embodiments, the plurality of polynucleotides comprises polynucleotides comprising nucleic acids collectively encoding at least 10 different variant gene products, at least 50 different variant gene products, at least 100 different variant gene products, at least 500 different variant gene products, at least 1,000 different variant gene products, at least 2000 different variant gene products, at least 5,000 different variant gene products, at least 10,000 different variant gene products, at least 20,000 different variant gene products, at least 30,000 different variant gene products, at least 50,000 different variant gene products, at least 75,000 different variant gene products, at least 100,000 different variant gene products, at least 200,000 different variant gene products, at least 300,000 different variant gene products, at least 500,000 different variant gene products, at least 1,000,000 different variant gene products, at least 10,000,000 different variant gene products, or at least 100,000,000 different variant gene products. In some embodiments, the plurality of polynucleotides comprises polynucleotides comprising nucleic acids collectively encoding 2-100,000 different variant gene products. In some embodiments, the plurality of polynucleotides comprises polynucleotides comprising nucleic acids collectively encoding 2-300,000 different variant gene products. In some embodiments, the plurality of polynucleotides comprises polynucleotides comprising nucleic acids collectively encoding 100-100,000 different variant gene products. In some embodiments, the plurality of polynucleotides comprises polynucleotides comprising nucleic acids collectively encoding 100-300,000 different variant gene products. In some embodiments, the plurality of polynucleotides comprises polynucleotides comprising nucleic acids collectively encoding 106 to 1010 different variant gene products.

In some embodiments, polynucleotides of the plurality of polynucleotides comprise nucleic acids encoding different variants of the same gene product. A “variant” refers to a variant of a gene (e.g., referring to a change in the nucleic acid sequence of the gene) and/or a variant of a gene product (e.g., a change in the amino acid sequence of a protein that is the gene product) depending on the context. A “gene variant” refers to a gene comprising a nucleotide change relative to a corresponding reference gene of the gene (e.g., a corresponding wild-type gene or a corresponding known variant of the gene). For example, the corresponding reference gene may encode a DNA polymerase sequence that has been modified to increase activity. In this example, the gene variant comprises at least one nucleotide change relative to the corresponding DNA polymerase sequence. In another example, the corresponding reference gene may be a wild-type DNA polymerase sequence. In some embodiments, the corresponding reference gene is KOD DNA polymerase. In some embodiments, the correspondence reference gene is Taq DNA polymerase. In some embodiments, a gene variant refers to a gene comprising a non-synonymous codon relative to a corresponding reference gene. In some embodiments, a gene variant refers to a gene comprising a synonymous codon relative to a corresponding reference gene. In some embodiments, a gene variant refers to a gene comprising an insertion or deletion relative to a wild-type gene.

A “gene product” refers to a protein or peptide encoded by the gene. In some embodiments, the gene product is a fusion protein. In some embodiments, the gene product comprises a localization or purification tag. In some embodiments, the gene product is an enzyme. In some embodiments, the gene product is a DNA modifying enzyme. In some embodiments, the gene product is an RNA modifying enzyme. In some embodiments, the gene product is a DNA polymerase. In some embodiments, the gene product is a Family A polymerase. Family A polymerases comprise a polymerase domain, a Klenow fold, a 5′ to 3′ exonuclease domain. In some embodiments, the 5′ to 3′ exonuclease domain is not required for polymerase function and is not included in recombinantly produced Family A polymerase. Family A polymerases are known in the art, e.g. as described in Czernecki et al. Nucleic Acids Research 51.9 (2023): 4488-4507. Family A polymerases include, but are not limited to T7 DNA polymerase, mitochondrial polymerase gamma, Taq polymerase, E. coli DNA polymerase I, and Bst polymerase.

In some embodiments, the gene product is a Family B polymerase. Family B polymerases are high fidelity polymerases and comprise 3′-5′ proofreading of newly synthesized DNA. Family B. polymerases include, but are not limited to, Pfu polymerase, phi29 DNA polymerase, E. coli DNA polymerase II, and KOD polymerase. Family B polymerases are known in the art, e.g., as described in Kazlauskas D Nucleic Acids Res. 2020 Oct. 9; 48 (18): 10142-10156.

In some embodiments, the gene product is a Taq polymerase or a Thermococcus kodakarensis (KOD) polymerase. “Variant(s) of the same gene product” or a “variant gene product(s)” refers to a gene product comprising an amino acid change relative to a corresponding reference gene of the gene product (e.g., a corresponding wild-type gene product or a corresponding known variant of the gene product).

A “codon” refers to a trinucleotide that encodes for an amino acid, a start codon, or a stop codon. A codon may be an RNA trinucleotide or a DNA trinucleotide. For example, the DNA trinucleotide TTT (UUU in an RNA) encodes for the amino acid phenylalanine. Additionally, it is known in the art that there is degeneracy in the mapping between codons and the 20 amino acids commonly encoded by codons. For example, both TTT and TTC encode for phenylalanine. Different codons that encode for the same amino acid are referred to as “synonymous codons”. Additionally, different codons that encode for a stop codon are also referred to as “synonymous codons”. When a polynucleotide comprises a “synonymous codon” at a given position of a gene this refers to a codon change in the given position of the gene that does not change the amino acid encoded by the given position. Different synonymous codons may have different frequencies of usage for a given organism. For example, in humans ATT, ATC, and ATA each encode isoleucine, but ATT encodes isoleucine 36% of the time, ATC encodes isoleucine 48% of the time, and ATA encodes isoleucine 16% of the time.

“Non-synonymous codons” refer to codons that encode different amino acids. When a polynucleotide comprises a non-synonymous codon at a given position (e.g., a first position) of a gene this refers to a codon change in the given position of the gene that changes the amino acid encoded by the given position. In some embodiments, non-synonymous codon changes in a polynucleotide are chosen such that the original codon (e.g., in the corresponding reference gene) and the non-synonymous codon have similar frequency of usage. In some embodiments, synonymous codon changes in a polynucleotide are chosen such that the original codon (e.g., in the corresponding reference gene) and the synonymous codon have similar usage. Codon usage tables for many different species are well known in the art.

The mapping between codons and the amino acids they encode is known in the art. Additionally, the mapping between codons and the amino acids they encode is highly conserved between species with very few exceptions. Table 1 provides an exemplary mapping between RNA codon sequences and encoded amino acids.

TABLE 1
RNA Codons

| | | | | | | | |
|---|---|---|---|---|---|---|---|
| UUU | (Phe/F) | UCU | (Ser/S) | UAU | (Tyr/Y) | UGU | (Cys/C) |
| UUC | Phenylalanine | UCC | Serine | UAC | Tyrosine | UGC | Cysteine |
| UUA | (Leu/L) | UCA | | UAA | Stop | UGA | Stop |
| UUG | Leucine | UCG | | UAG | Stop | UGG | (Trp/W) |
| | | | | | | | Tryptophan |
| CUU | | CCU | (Pro/P) | CAU | (His/H) | CGU | (Arg/R) |
| CUC | | CCC | Proline | CAC | Histidine | CGC | Arginine |
| CUA | | CCA | | CAA | (Gln/Q) | CGA | |
| CUG | | CCG | | CAG | Glutamine | CGG | |
| AUU | (Ile/I) | ACU | (Thr/T) | AAU | (Asn/N) | AGU | (Ser/S) |
| AUC | Isoleucine | ACC | Threonine | AAC | Asparagine | AGC | Serine |
| AUA | | ACA | | AAA | (Lys/K) | AGA | (Arg/R) |
| AUG | (Met/M) | ACG | | AAG | Lysine | AGG | Arginine |
| | Methionine | | | | | | |
| | (start) | | | | | | |
| GUU | (Val/V) | GCU | (Ala/A) | GAU | (Asp/D) | GGU | (Gly/G) |
| GUC | Valine | GCC | Alanine | GAC | Aspartic acid | GGC | Glycine |
| GUA | | GCA | | GAA | (Glu/E) | GGA | |
| GUG | | GCG | | GAG | Glutamic acid | GGG | |

### First Polynucleotide

In some embodiments, a plurality of polynucleotides of a library comprises a first polynucleotide that comprises nucleic acids encoding a first variant, the nucleic acids comprising a first non-synonymous codon at a first position and a synonymous codon (e.g., a first synonymous codon).

The first position refers to the location of any one codon in the nucleic acids encoding the first variant. The first position can be used as a reference point between different polynucleotides comprising nucleic acids encoding different variants of the same gene product. For example, the first position refers to the same position in: the first polynucleotide comprising nucleic acids encoding the first variant, the second polynucleotide comprising the nucleic acids encoding the second variant, the third polynucleotide comprising the nucleic acids encoding the third variant, the fourth polynucleotide comprising nucleic acids encoding the fourth variant, and the additional polynucleotides comprising nucleic acids encoding additional variants.

The first non-synonymous codon at the first position changes the amino acid encoded at the first position (e.g., relative to the corresponding reference gene) to any other amino acid or to a stop codon. For example, the non-synonymous codon at the first position may change a codon encoding a phenylalanine to a codon encoding any other amino acid in Table 1. The synonymous codon may be at any position in the nucleic acids encoding the first variant except for the first position.

In some embodiments, the nucleic acids of the first polynucleotide comprise an additional synonymous codon. The additional synonymous codon may be at any position of the nucleic acids encoding the first variant except the first position and the position of the synonymous codon (i.e., the first synonymous codon). The additional synonymous codon may be added to increase the Hamming distance between the nucleic acids encoding the first variant and the nucleic acids encoding the other variants in the plurality of polynucleotides to at least 2.

In some embodiments, the non-synonymous codon at the first position, the synonymous codon, and optionally the additional synonymous codon are within about 10,000 nucleotides of one another in the first polynucleotide. In some embodiments, the non-synonymous codon at the first position, the synonymous codon, and optionally the additional synonymous codon are within about 5,000 nucleotides of one another in the first polynucleotide. In some embodiments, the non-synonymous codon at the first position, the synonymous codon, and optionally the additional synonymous codon are within about 1,000 nucleotides of one another in the first polynucleotide. In some embodiments, the non-synonymous codon at the first position, the synonymous codon, and optionally the additional synonymous codon are within about 500 nucleotides of one another in the first polynucleotide. In some embodiments, the non-synonymous codon at the first position, the synonymous codon, and optionally the additional synonymous codon are within about 200 nucleotides of one another in the first polynucleotide. In some embodiments, the non-synonymous codon at the first position, the synonymous codon, and optionally the additional synonymous codon are within about 100 nucleotides of one another in the first polynucleotide. In some embodiments, the non-synonymous codon at the first position, the synonymous codon, and optionally the additional synonymous codon are within about 50 nucleotides of one another in the first polynucleotide. In some embodiments, the non-synonymous codon at the first position, the synonymous codon, and optionally the additional synonymous codon are within about 30 nucleotides of one another in the first polynucleotide. In some embodiments, the non-synonymous codon at the first position, the synonymous codon, and optionally the additional synonymous codon are within about 10 nucleotides of one another in the first polynucleotide. In some embodiments, the non-synonymous codon at the first position, the synonymous codon, and optionally the additional synonymous codon are within 3 nucleotides of one another in the first polynucleotide. In some embodiments, the non-synonymous codon at the first position, the synonymous codon, and optionally the additional synonymous codon are adjacent to one another in the first polynucleotide. For example, in the polynucleotide sequence . . . UUU-UUA-AUA-AUG (SEQ ID NO: 1) . . . UUA and AUA are adjacent to one another. UUU and UUA are also adjacent to one another. UUU UUA and AUA are adjacent to one another. UUU and AUA are not adjacent to one another. In some embodiments, the order of adjacency in the first polynucleotide is synonymous codon-non-synonymous codon-additional synonymous codon in consecutive order. This is also referred to as a super codon.

### Second Polynucleotide

In some embodiments, a plurality of polynucleotides of a library comprises a second polynucleotide comprising nucleic acids encoding a second variant, the nucleic acids comprising a second non-synonymous codon at the first position and a synonymous codon (e.g., a second synonymous codon). The second non-synonymous codon at the first position changes the amino acid encoded at the first position (e.g., relative to the corresponding reference gene) to any other amino acid or to a stop codon except the amino acid or stop codon encoded by the first non-synonymous codon in the first polynucleotide. The synonymous codon may be at any position in the nucleic acids encoding the second variant except for the first position.

In some embodiments, the synonymous codon in the first polynucleotide and the synonymous codon in the second polynucleotide are at different positions. In some embodiments, the synonymous codon in the first polynucleotide and the synonymous codon in the second polynucleotide are at the same position. In some embodiments, the synonymous codon in the first polynucleotide and the synonymous codon in the second polynucleotide are at the same position and are different synonymous codons. In some embodiments, the synonymous codon in the first polynucleotide and the synonymous codon in the second polynucleotide are at the same position and are the same synonymous codons. In some embodiments, the synonymous codon in the first polynucleotide and the synonymous codon in the second polynucleotide are selected to increase the Hamming distance to at least 2 between the first polynucleotide and the second polynucleotide.

In some embodiments, the second polynucleotide comprises an additional synonymous codon. The additional synonymous codon may be at any position of the second variant except the first position and the position of the synonymous codon (i.e., the first synonymous codon in the second polynucleotide). The additional synonymous codon may be added to increase the Hamming distance to at least 2 between the nucleic acids encoding the second variant and the nucleic acids encoding other variants in the plurality of polynucleotides. The additional synonymous codon may be added to increase the Hamming distance to at least 2 between the nucleic acids encoding the second variant and the nucleic acids encoding the other variants in the plurality of polynucleotide with a non-synonymous codon at position 1.

In some embodiments, the non-synonymous codon at the first position, the synonymous codon, and optionally the additional synonymous codon are within about 10,000 nucleotides of one another in the second polynucleotide. In some embodiments, the non-synonymous codon at the first position, the synonymous codon, and optionally the additional synonymous codon are within about 5,000 nucleotides of one another in the second polynucleotide. In some embodiments, the non-synonymous codon at the first position, the synonymous codon, and optionally the additional synonymous codon are within about 1,000 nucleotides of one another in the second polynucleotide. In some embodiments, the non-synonymous codon at the first position, the synonymous codon, and optionally the additional synonymous codon are within about 500 nucleotides of one another in the second polynucleotide. In some embodiments, the non-synonymous codon at the first position, the synonymous codon, and optionally the additional synonymous codon are within about 200 nucleotides of one another in the second polynucleotide. In some embodiments, the non-synonymous codon at the first position, the synonymous codon, and optionally the additional synonymous codon are within about 100 nucleotides of one another in the second polynucleotide. In some embodiments, the non-synonymous codon at the first position, the synonymous codon, and optionally the additional synonymous codon are within about 50 nucleotides of one another in the second polynucleotide. In some embodiments, the non-synonymous codon at the first position, the synonymous codon, and optionally the additional synonymous codon are within about 30 nucleotides of one another in the second polynucleotide. In some embodiments, the non-synonymous codon at the first position, the synonymous codon, and optionally the additional synonymous codon are within about 10 nucleotides of one another in the second polynucleotide. In some embodiments, the non-synonymous codon at the first position, the synonymous codon, and optionally the additional synonymous codon are within 3 nucleotides of one another in the second polynucleotide. In some embodiments, the non-synonymous codon at the first position, the synonymous codon, and optionally the additional synonymous codon are adjacent to one another in the second polynucleotide. In some embodiments, the order of adjacency in the second polynucleotide is synonymous codon-non-synonymous codon-additional synonymous codon in consecutive order. This is also referred to as a super codon.

In some embodiments, plurality of polynucleotides comprises: (i) a first polynucleotide encoding a first variant, wherein the first polynucleotide comprises a first non-synonymous codon and a synonymous codon (e.g., as described herein); and (ii) a second polynucleotide encoding a second variant, wherein the second polynucleotide comprises a second non-synonymous codon and a synonymous codon (e.g., as described herein).

### Third Polynucleotide

In some embodiments, a plurality of polynucleotides of a library comprises a third polynucleotide, wherein the third polynucleotide comprises nucleic acids encoding a third variant, the nucleic acids comprising: (i) a non-synonymous codon at the first position that is different from the first non-synonymous codon and the second non-synonymous codon; and (ii) a synonymous codon (e.g., a third synonymous codon). The third non-synonymous codon at the first position changes the amino acid encoded at the first position (e.g., relative to the corresponding reference gene) to any other amino acid or a stop codon except the amino acid or stop codon encoded by the first non-synonymous codon in the first polynucleotide and the second non-synonymous codon in the second polynucleotide. The synonymous codon may be at any position in the nucleic acids encoding the third variant except for the first position.

In some embodiments, the synonymous codon in the first polynucleotide and/or the synonymous codon in the second polynucleotide, and the synonymous codon in the third polynucleotide are at different positions. In some embodiments, the synonymous codon in the first polynucleotide and/or the synonymous codon in the second polynucleotide and the synonymous codon in the third polynucleotide are at the same position. In some embodiments, the synonymous codon in the first polynucleotide and/or the synonymous codon in the second polynucleotide and the synonymous codon in the third polynucleotide are at the same position and are different synonymous codons. In some embodiments, the synonymous codon in the first polynucleotide and/or the synonymous codon in the second polynucleotide and the synonymous codon in the third polynucleotide are at the same position and are the same synonymous codons. In some embodiments, the synonymous codon in the first polynucleotide, the synonymous codon in the second polynucleotide, and the synonymous codon in the third polynucleotide are selected to increase the Hamming distance to at least 2 between the first polynucleotide, the second polynucleotide, and the third polynucleotide.

In some embodiments, the nucleic acids of the third polynucleotide comprise an additional synonymous codon. The additional synonymous codon may be at any position in the nucleic acids encoding the third variant except the first position and the position of the synonymous codon (i.e., the first synonymous codon in the third polynucleotide). The additional synonymous codon may be added to increase the Hamming distance to at least 2 between the nucleic acids encoding the third variant and the nucleic acids encoding the other variants in the plurality of polynucleotides. The additional synonymous codon may be added to increase the Hamming distance to at least 2 between the nucleic acids encoding the third variant and the nucleic acids encoding the variants in the plurality of polynucleotides with a non-synonymous codon at position 1.

In some embodiments, the non-synonymous codon at the first position, the synonymous codon, and optionally the additional synonymous codon are within about 15,000 nucleotides of one another in the third polynucleotide. “About” refers to within 5%, e.g., within 5%, 4%, 3%, 2%, or 1% of a given value or range. In some embodiments, the non-synonymous codon at the first position, the synonymous codon, and optionally the additional synonymous codon are within about 10,000 nucleotides of one another in the third polynucleotide. In some embodiments, the non-synonymous codon at the first position, the synonymous codon, and optionally the additional synonymous codon are within about 5,000 nucleotides of one another in the third polynucleotide. In some embodiments, the non-synonymous codon at the first position, the synonymous codon, and optionally the additional synonymous codon are within about 1,000 nucleotides of one another in the third polynucleotide. In some embodiments, the non-synonymous codon at the first position, the synonymous codon, and optionally the additional synonymous codon are within about 500 nucleotides of one another in the third polynucleotide. In some embodiments, the non-synonymous codon at the first position, the synonymous codon, and optionally the additional synonymous codon are within about 200 nucleotides of one another in the third polynucleotide. In some embodiments, the non-synonymous codon at the first position, the synonymous codon, and optionally the additional synonymous codon are within about 100 nucleotides of one another in the third polynucleotide. In some embodiments, the non-synonymous codon at the first position, the synonymous codon, and optionally the additional synonymous codon are within about 50 nucleotides of one another in the third polynucleotide. In some embodiments, the non-synonymous codon at the first position, the synonymous codon, and optionally the additional synonymous codon are within about 30 nucleotides of one another in the third polynucleotide. In some embodiments, the non-synonymous codon at the first position, the synonymous codon, and optionally the additional synonymous codon are within about 10 nucleotides of one another in the third polynucleotide. In some embodiments, the non-synonymous codon at the first position, the synonymous codon, and optionally the additional synonymous codon are within 3 nucleotides of one another in the third polynucleotide. In some embodiments, the non-synonymous codon at the first position, the synonymous codon, and optionally the additional synonymous codon are adjacent to one another in the third polynucleotide. In some embodiments, the order of adjacency in the third polynucleotide is synonymous codon-non-synonymous codon-additional synonymous codon in consecutive order. This is also referred to as a super codon.

In some embodiments, the first polynucleotide, the second polynucleotide and the third polynucleotide comprise different non-synonymous codons at the first position, and the first position is adjacent to the synonymous codon and the additional synonymous codon. For example, the first polynucleotide, the second polynucleotide and the third polynucleotide may comprise a sequence according to the following:

| | −1 position | First position | +1 position |
|---|---|---|---|
| Reference Sequence | UUA (Leu) | UCU (Ser) | CGU (Arg) |
| First polynucleotide | CUU (Leu) | UAU (Tyr) | CGC (Arg) |
| Second polynucleotide | CUU (Leu) | UAA (Stop) | CGA (Arg) |
| Third polynucleotide | CUC (Leu) | UGG (Trp) | CGA (Arg) |

In some embodiments, the first polynucleotide, the second polynucleotide and the third polynucleotide differ by a Hamming distance of at least 2.

### Fourth Polynucleotide

In some embodiments, a library comprising a plurality of polynucleotides comprises a fourth polynucleotide, wherein the fourth polynucleotide comprises nucleic acids encoding a fourth variant, the nucleic acids comprising (i) a non-synonymous codon at a different position than the first position and (ii) a synonymous codon (e.g., the fourth synonymous codon). The non-synonymous codon at the different position from the first position is located at any other position in the nucleic acids encoding the fourth variant except the first position. The non-synonymous codon may be any non-synonymous codon including a stop codon. The synonymous codon is at any position in the nucleic acids encoding the fourth variant except the different position.

In some embodiments, the fourth polynucleotide comprises an additional synonymous codon. The additional synonymous codon is at any position in the nucleic acids encoding the fourth variant except the different position and the position of the synonymous codon (i.e., the first synonymous codon in the fourth variant). The additional synonymous codon may be added to increase the Hamming distance between the fourth variant and the other variants in the plurality of polynucleotides to at least 2.

In some embodiments, the non-synonymous codon at the different position, the synonymous codon, and optionally the additional synonymous codon are within about 15,000 nucleotides of one another in the fourth polynucleotide. In some embodiments, the non-synonymous codon at the different position, the synonymous codon, and optionally the additional synonymous codon are within about 10,000 nucleotides of one another in the fourth polynucleotide. In some embodiments, the non-synonymous codon at the different position, the synonymous codon, and optionally the additional synonymous codon are within about 5,000 nucleotides of one another in the fourth polynucleotide. In some embodiments, the non-synonymous codon at the different position, the synonymous codon, and optionally the additional synonymous codon are within about 1,000 nucleotides of one another in the fourth polynucleotide. In some embodiments, the non-synonymous codon at the different position, the synonymous codon, and optionally the additional synonymous codon are within about 500 nucleotides of one another in the fourth polynucleotide. In some embodiments, the non-synonymous codon at the different position, the synonymous codon, and optionally the additional synonymous codon are within about 200 nucleotides of one another in the fourth polynucleotide. In some embodiments, the non-synonymous codon at the different position, the synonymous codon, and optionally the additional synonymous codon are within about 100 nucleotides of one another in the fourth polynucleotide. In some embodiments, the non-synonymous codon at the different position, the synonymous codon, and optionally the additional synonymous codon are within about 50 nucleotides of one another in the fourth polynucleotide. In some embodiments, the non-synonymous codon at the different position, the synonymous codon, and optionally the additional synonymous codon are within about 30 nucleotides of one another in the fourth polynucleotide. In some embodiments, the non-synonymous codon at the different position, the synonymous codon, and optionally the additional synonymous codon are within about 10 nucleotides of one another in the fourth polynucleotide. In some embodiments, the non-synonymous codon at the different position, the synonymous codon, and optionally the additional synonymous codon are within 3 nucleotides of one another in the fourth polynucleotide. In some embodiments, the non-synonymous codon at the different position, the synonymous codon, and optionally the additional synonymous codon are adjacent to one another in the fourth polynucleotide. In some embodiments, the order of adjacency in the fourth polynucleotide is synonymous codon-non-synonymous codon-additional synonymous codon in consecutive order. This is also referred to as a super codon.

### Additional Polynucleotides

In some embodiments, a plurality of polynucleotide of a library further comprises additional polynucleotides that each comprise nucleic acids encoding a variant gene product. In some embodiments, most of the additional polynucleotides comprise a gene variant comprising: (i) a non-synonymous codon; and (ii) a synonymous codon. In some embodiments, each of the additional polynucleotides comprises a gene variant comprising: (i) a non-synonymous codon; and (ii) a synonymous codon. In some embodiments, each of the additional polynucleotides comprises a gene variant comprising: (i) a non-synonymous codon; and (ii) a synonymous codon, as needed to increase the Hamming distance of the additional polynucleotides form other polynucleotides to at least 2. The two non-synonymous codons mentioned in the additional polynucleotides mentioned here are in the nucleic acids encoding the gene variant. The synonymous codons in the additional polynucleotides are in the nucleic acids encoding the gene variant.

In some embodiments, the nucleic acids of polynucleotides of the additional polynucleotides comprise the non-synonymous codon at different positions. For example, one polynucleotide may comprise the non-synonymous codon at nucleic acids 10-12 of a gene variant, whereas another polynucleotide may comprise the non-synonymous codon at nucleic acids 16-18 of a gene different variant. In some embodiments, polynucleotides of the additional polynucleotides comprise the non-synonymous codon at the same position. For example, one polynucleotide may comprise the non-synonymous codon at nucleic acids 10-12 of a gene variant, and another polynucleotide may also comprise the non-synonymous codon at nucleic acids 10-12 of a different gene variant.

In some embodiments, the nucleic acids of the polynucleotides of the additional polynucleotides comprise the synonymous codon at different positions. For example, one polynucleotide may comprise the synonymous codon at nucleic acids 10-12 of a gene variant, whereas another polynucleotide may comprise the synonymous codon at nucleic acids 16-18 of a different gene variant. In some embodiments, polynucleotides of the additional polynucleotides comprise the synonymous codon at the same position. For example, one polynucleotide may comprise the synonymous codon at nucleic acids 10-12 of a gene variant, and another polynucleotide may also comprise the synonymous codon at nucleic acids 10-12 of a different gene variant.

In some embodiments, the nucleic acids of the one or more of the additional polynucleotides comprises an additional synonymous codon. The additional synonymous codon may be in a given additional polynucleotide at any position in the nucleic acids encoding the variant except the position of the non-synonymous codon and position of the synonymous codon (e.g., the synonymous codon on the given additional polynucleotide). In some embodiments, the polynucleotides of the one or more additional polynucleotides comprise the additional synonymous codon at different positions in the nucleic acids encoding the variant. For example, a first additional polynucleotide comprises the additional synonymous codon at position 12 and the second additional polynucleotide comprises the additional synonymous codon at position 92.

In some embodiments, the non-synonymous codon, the synonymous codon, and optionally the additional synonymous codon are within about 15,000 nucleotides of one another in most of the one or more additional polynucleotides. In some embodiments, the non-synonymous codon, the synonymous codon, and optionally the additional synonymous codon are within about 15,000 nucleotides of one another in each of the one or more additional polynucleotides.

In some embodiments, the non-synonymous codon, the synonymous codon, and optionally the additional synonymous codon are within about 10,000 nucleotides of one another in most of the one or more additional polynucleotides. In some embodiments, the non-synonymous codon, the synonymous codon, and optionally the additional synonymous codon are within about 10,000 nucleotides of one another in each of the one or more additional polynucleotides.

In some embodiments, the non-synonymous codon, the synonymous codon, and optionally the additional synonymous codon are within about 5,000 nucleotides of one another in most of the one or more additional polynucleotides. In some embodiments, the non-synonymous codon, the synonymous codon, and optionally the additional synonymous codon are within about 5,000 nucleotides of one another in each of the one or more additional polynucleotides.

In some embodiments, the non-synonymous codon, the synonymous codon, and optionally the additional synonymous codon are within about 1,000 nucleotides of one another in most of the one or more additional polynucleotides. In some embodiments, the non-synonymous codon, the synonymous codon, and optionally the additional synonymous codon are within about 1,000 nucleotides of one another in each of the one or more additional polynucleotides.

In some embodiments, the non-synonymous codon, the synonymous codon, and optionally the additional synonymous codon are within about 500 nucleotides of one another in most of the one or more additional polynucleotides. In some embodiments, the non-synonymous codon, the synonymous codon, and optionally the additional synonymous codon are within about 500 nucleotides of one another in each of the one or more additional polynucleotides.

In some embodiments, the non-synonymous codon, the synonymous codon, and optionally the additional synonymous codon are within about 200 nucleotides of one another in most of the one or more additional polynucleotides. In some embodiments, the non-synonymous codon, the synonymous codon, and optionally the additional synonymous codon are within about 200 nucleotides of one another in each of the one or more additional polynucleotides.

In some embodiments, the non-synonymous codon, the synonymous codon, and optionally the additional synonymous codon are within about 100 nucleotides of one another in most of the one or more additional polynucleotides. In some embodiments, the non-synonymous codon, the synonymous codon, and optionally the additional synonymous codon are within about 100 nucleotides of one another in each of the one or more additional polynucleotides.

In some embodiments, the non-synonymous codon, the synonymous codon, and optionally the additional synonymous codon are within about 50 nucleotides of one another in most of the one or more additional polynucleotides. In some embodiments, the non-synonymous codon, the synonymous codon, and optionally the additional synonymous codon are within about 50 nucleotides of one another in each of the one or more additional polynucleotides.

In some embodiments, the non-synonymous codon, the synonymous codon, and optionally the additional synonymous codon are within about 30 nucleotides of one another in most of the one or more additional polynucleotides. In some embodiments, the non-synonymous codon, the synonymous codon, and optionally the additional synonymous codon are within about 30 nucleotides of one another in each of the one or more additional polynucleotides.

In some embodiments, the non-synonymous codon, the synonymous codon, and optionally the additional synonymous codon are within about 10 nucleotides of one another in most of the one or more additional polynucleotides. In some embodiments, the non-synonymous codon, the synonymous codon, and optionally the additional synonymous codon are within about 10 nucleotides of one another in each of the one or more additional polynucleotides.

In some embodiments, the non-synonymous codon, the synonymous codon, and optionally the additional synonymous codon are within about 10 nucleotides of one another in most of the one or more additional polynucleotides. In some embodiments, the non-synonymous codon, the synonymous codon, and optionally the additional synonymous codon are within about 10 nucleotides of one another in each of the one or more additional polynucleotides.

In some embodiments, the non-synonymous codon, the synonymous codon, and optionally the additional synonymous codon are within 3 nucleotides of one another in most of the one or more additional polynucleotides. In some embodiments, the non-synonymous codon, the synonymous codon, and optionally the additional synonymous codon are within 3 nucleotides of one another in each of the one or more additional polynucleotides.

In some embodiments, the non-synonymous codon, the synonymous codon, and optionally the additional synonymous codon are adjacent to one another in most of the one or more additional polynucleotides. In some embodiments, the non-synonymous codon, the synonymous codon, and optionally the additional synonymous codon are adjacent to one another in each of the one or more additional polynucleotides.

In some embodiments, the order of adjacency in most of the one or more additional polynucleotides is synonymous codon-non-synonymous codon-additional synonymous codon in consecutive order. In some embodiments, the order of adjacency in the one or more additional polynucleotides is synonymous codon-non-synonymous codon-additional synonymous codon in consecutive order. This is also referred to as a super codon.

In some embodiments, at least 50% of the first polynucleotide, the second polynucleotide, the third polynucleotide, the fourth polynucleotide and the additional polynucleotides differ by a Hamming distance of at least 2. In some embodiments, at least 90% of the first polynucleotide, the second polynucleotide, the third polynucleotide, the fourth polynucleotide and the additional polynucleotides differ by a Hamming distance of at least 2. In some embodiments, at least 95% of the first polynucleotide, the second polynucleotide, the third polynucleotide, the fourth polynucleotide and the additional polynucleotides differ by a Hamming distance of at least 2. In some embodiments, 100% of the first polynucleotide, the second polynucleotide, the third polynucleotide, the fourth polynucleotide and the additional polynucleotides differ by a Hamming distance of at least 2.

In some embodiments, the first polynucleotide, the second polynucleotide, the third polynucleotide and the additional polynucleotide collectively encode at least 25% of possible amino acid substitutions at the first position. “Possible amino acid substitutions” refer to amino acid substitutions to the 20 common amino acids (e.g., as listed in Table 1). In some embodiments, the first polynucleotide, the second polynucleotide, the third polynucleotide and the additional polynucleotides collectively encode at least 50% of possible amino acid substitutions at the first position. In some embodiments, the first polynucleotide, the second polynucleotide, and the additional polynucleotides collectively encode at least 75% of possible amino acid substitutions at the first position. In some embodiments, the first polynucleotide, the second polynucleotide, and the additional polynucleotides collectively encode at least 90% of possible amino acid substitutions at the first position. In some embodiments, the first polynucleotide, the second polynucleotide, and the additional polynucleotides collectively encode 100% of possible amino acid substitutions at the first position. In some embodiments, the first polynucleotide, the second polynucleotide, and the additional polynucleotides collectively encode all hydrophobic amino acid substitutions at the first position (e.g., A, V, I, L, M, F, Y and W). In some embodiments, the first polynucleotide, the second polynucleotide, and the additional polynucleotides collectively encode all polar amino acid substitutions at the first position (e.g., R, H, K, D, E, S, T, N, and Q). In some embodiments, the first polynucleotide, the second polynucleotide, and the additional polynucleotide collectively encode all negatively charged amino acid substitutions at the first position (e.g., D and E). In some embodiments, the first polynucleotide, the second polynucleotide, and the additional polynucleotide collectively encode all positively charged amino acid substitutions at the first position (e.g., R, H, and K). In some embodiments, the first polynucleotide, the second polynucleotide, and the additional polynucleotides collectively encode all bulky amino acid substitutions at the first position (e.g., F, Y, and W, and optionally H).

In some embodiments, the first polynucleotide, the second polynucleotide, the third polynucleotide, the fourth polynucleotide, and the additional polynucleotides collectively encode at least 10% of possible single amino acid substitutions of the gene product. In some embodiments, the first polynucleotide, the second polynucleotide, the third polynucleotide, the fourth polynucleotide, and the additional polynucleotides collectively encode at least 25% of possible single amino acid substitutions of the gene product. In some embodiments, the first polynucleotide, the second polynucleotide, the third polynucleotide, the fourth polynucleotide, and the additional polynucleotides collectively encode at least 50% of possible single amino acid substitutions of the gene product. In some embodiments, the first polynucleotide, the second polynucleotide, the third polynucleotide, the fourth polynucleotide, and the additional polynucleotides collectively encode at least 75% of possible single amino acid substitutions of the gene product. In some embodiments, the first polynucleotide, the second polynucleotide, the third polynucleotide, the fourth polynucleotide, and the additional polynucleotides collectively encode at least 85% of possible single amino acid substitutions of the gene product. In some fourth polynucleotide, and the additional polynucleotides collectively encode at least 90% of possible single amino acid substitutions of the gene product. In some embodiments, the first polynucleotide, the second polynucleotide, the third polynucleotide, the fourth polynucleotide, and the additional polynucleotides collectively encode at least 95% of possible single amino acid substitutions of the gene product. In some embodiments, the first polynucleotide, the second polynucleotide, the third polynucleotide, the fourth polynucleotide, and the additional polynucleotides collectively encode at least 98% of possible single amino acid substitutions of the gene product. In some embodiments, the first polynucleotide, the second polynucleotide, the third polynucleotide, the fourth polynucleotide, and the additional polynucleotides collectively encode at least 99% of possible single amino acid substitutions of the gene product. In some embodiments, the first polynucleotide, the second polynucleotide, the third polynucleotide, the fourth polynucleotide, and the additional polynucleotides collectively encode 100% of possible single amino acid substitutions of the gene product.

In some embodiments, the first polynucleotide, the second polynucleotide, the third polynucleotide, the fourth polynucleotide, and the additional polynucleotides collectively encode at least 10% of the possible single non-synonymous codons in the gene product. In some embodiments, the first polynucleotide, the second polynucleotide, the third polynucleotide, the fourth polynucleotide, and the additional polynucleotides collectively encode at least 25% of the possible single non-synonymous codons in the gene product. In some embodiments, the first polynucleotide, the second polynucleotide, the third polynucleotide, the fourth polynucleotide, and the additional polynucleotides collectively encode at least 50% of the possible single non-synonymous codons in the gene product. In some embodiments, the first polynucleotide, the second polynucleotide, the third polynucleotide, the fourth polynucleotide, and the additional polynucleotides collectively encode at least 75% of the possible single non-synonymous codons in the gene product. In some embodiments, the first polynucleotide, the second polynucleotide, the third polynucleotide, the fourth polynucleotide, and the additional polynucleotides collectively encode at least 90% of the possible single non-synonymous codons in the gene product. In some embodiments, the first polynucleotide, the second polynucleotide, the third polynucleotide, the fourth polynucleotide, and the additional polynucleotides collectively encode at least 95% of the possible single non-synonymous codons in the gene product. In some fourth polynucleotide, and the additional polynucleotides collectively encode at least 98% of the possible single non-synonymous codons in the gene product. In some embodiments, the first polynucleotide, the second polynucleotide, the third polynucleotide, the fourth polynucleotide, and the additional polynucleotides collectively encode at least 99% of the possible single non-synonymous codons in the gene product. In some embodiments, the first polynucleotide, the second polynucleotide, the third polynucleotide, the fourth polynucleotide, and the additional polynucleotides collectively encode 100% of the possible single non-synonymous codons in the gene product.

In some embodiments, the first polynucleotide, the second polynucleotide, the third polynucleotide, the fourth polynucleotide, and the additional polynucleotides collectively encode at least 50 different variants of the same gene product. In some embodiments, the first polynucleotide, the second polynucleotide, the third polynucleotide, the fourth polynucleotide, and the additional polynucleotides collectively encode at least 100 different variants of the same gene product. In some embodiments, the first polynucleotide, the second polynucleotide, the third polynucleotide, the fourth polynucleotide, and the additional polynucleotides collectively encode at least 250 different variants of the same gene product. In some embodiments, the first polynucleotide, the second polynucleotide, the third polynucleotide, the fourth polynucleotide, and the additional polynucleotides collectively encode at least 500 different variants of the same gene product. In some embodiments, the first polynucleotide, the second polynucleotide, the third polynucleotide, the fourth polynucleotide, and the additional polynucleotides collectively encode at least 1,000 different variants of the same gene product. In some embodiments, the first polynucleotide, the second polynucleotide, the third polynucleotide, the fourth polynucleotide, and the additional polynucleotides collectively encode at least 2,000 different variants of the same gene product. In some embodiments, the first polynucleotide, the second polynucleotide, the third polynucleotide, the fourth polynucleotide, and the additional polynucleotides collectively encode at least 3,000 different variants of the same gene product. In some embodiments, the first polynucleotide, the second polynucleotide, the third polynucleotide, the fourth polynucleotide, and the additional polynucleotides collectively encode at least 4,000 different variants of the same gene product. In some embodiments, the first polynucleotide, the second polynucleotide, the third polynucleotide, the fourth polynucleotide, and the additional polynucleotides collectively encode at least 5,000 different variants of the same gene product. In some embodiments, the first polynucleotide, the second polynucleotide, the third polynucleotide, the fourth polynucleotide, and the additional polynucleotides collectively encode at least 7,500 different variants of the same gene product. In some embodiments, the first polynucleotide, the second polynucleotide, the third polynucleotide, the fourth polynucleotide, and the additional polynucleotides collectively encode at least 10,000 different variants of the same gene product. In some embodiments, the first polynucleotide, the second polynucleotide, the third polynucleotide, the fourth polynucleotide, and the additional polynucleotides collectively encode at least 15,000 different variants of the same gene product. In some embodiments, the first polynucleotide, the second polynucleotide, the third polynucleotide, the fourth polynucleotide, and the additional polynucleotides collectively encode at least 20,000 different variants of the same gene product. In some embodiments, the first polynucleotide, the second polynucleotide, the third polynucleotide, the fourth polynucleotide, and the additional polynucleotides collectively encode at least 30,000 different variants of the same gene product. In some embodiments, the first polynucleotide, the second polynucleotide, the third polynucleotide, the fourth polynucleotide, and the additional polynucleotides collectively encode at least 50,000 different variants of the same gene product. In some embodiments, the first polynucleotide, the second polynucleotide, the third polynucleotide, the fourth polynucleotide, and the additional polynucleotides collectively encode at least 100,000 different variants of the same gene product.

In some embodiments, the first polynucleotide, the second polynucleotide, the third polynucleotide, the fourth polynucleotide, and the additional polynucleotides collectively encode 19-100,000 different variants of the same gene product. In some embodiments, the first polynucleotide, the second polynucleotide, the third polynucleotide, the fourth polynucleotide, and the additional polynucleotides collectively encode 50-100,000 different variants of the same gene product. In some embodiments, the first polynucleotide, the second polynucleotide, the third polynucleotide, the fourth polynucleotide, and the additional polynucleotides collectively encode 100-100,000 different variants of the same gene product. In some embodiments, the first polynucleotide, the second polynucleotide, the third polynucleotide, the fourth polynucleotide, and the additional polynucleotides collectively encode 50-30,000 different variants of the same gene product. In some embodiments, the first polynucleotide, the second polynucleotide, the third polynucleotide, the fourth polynucleotide, and the additional polynucleotides collectively encode 50-10,000 different variants of the same gene product. In some embodiments, the first polynucleotide, the second polynucleotide, the third polynucleotide, the fourth polynucleotide, and the additional polynucleotides collectively encode 100-10,000 different variants of the same gene product.

In some embodiments, each polynucleotide of the plurality of polynucleotides (e.g., the first polynucleotide, the second polynucleotide, the third polynucleotide, the fourth polynucleotide and/or the additional polynucleotide) encode a single variant.

### Synonymous Polynucleotide

In some embodiments, the library comprises a synonymous polynucleotide. A “synonymous polynucleotide” refers to a polynucleotide comprising nucleic acids encoding a gene variant that comprises a synonymous codon and does not change the amino acid sequence encoded by the gene. In some embodiments, the nucleic acids of the synonymous polynucleotides are modified to have a Hamming distance of 2 or more from other polynucleotides in the plurality of polynucleotides. In some embodiments, the synonymous polynucleotide comprises 1, 2 or 3 synonymous codons. The different synonymous codons may be within 200 nucleotides, 100 nucleotides, 50 nucleotides, 30 nucleotides, 10 nucleotides or 3 nucleotides of one another in the synonymous polynucleotide. In some embodiments, the different synonymous codons are adjacent to one another in the synonymous polynucleotide. For example, a synonymous polynucleotide can comprise two adjacent synonymous codons. In another example, a synonymous polynucleotide can comprise three adjacent synonymous codons (e.g., a super codon of synonymous codons). In some embodiments, the synonymous polynucleotide encodes the amino acid sequence of the reference sequence. In some embodiments, the synonymous polynucleotide encodes the amino acid sequence of the wild-type version of the gene for which variants are being made. In some embodiments, the wild-type version of a gene is a naturally occurring version of the gene.

In some embodiments, this disclosure provides a plurality of synonymous polynucleotides. In some embodiments, the plurality of polynucleotides comprises a plurality of synonymous polynucleotides. In some embodiments, the plurality of synonymous polynucleotides comprises a synonymous codon in at least 1 (e.g., at least 2, at least 5, at least 10, at least 25, at least 25, at least 50, at least 100, at least 200, at least 500 positions) position of the gene. In some embodiments, the plurality of synonymous polynucleotides comprises a synonymous codon in at least 10% (at least 10%, at least 20%, at least 30%, at least 40%, at least 50%, at least 60%, at least 70%, at least 80%, at least 90%, at least 95%, at least 98%, or at least 99%) of positions in the gene. In some embodiments, the plurality of synonymous polynucleotides comprises a synonymous codon at each position of the gene.

In some embodiments, at least 50% of the first polynucleotide, the second polynucleotide, the third polynucleotide, the fourth polynucleotide, and the additional polynucleotides differ by a Hamming distance of at least 2. In some embodiments, at least 90% of the first polynucleotide, the second polynucleotide, the third polynucleotide, the fourth polynucleotide, and the additional polynucleotides differ by a Hamming distance of at least 2. In some embodiments, at least 95% of the first polynucleotide, the second polynucleotide, the third polynucleotide, the fourth polynucleotide, and the additional polynucleotides differ by a Hamming distance of at least 2. In some embodiments, at 100% of the first polynucleotide, the second polynucleotide, the third polynucleotide, the fourth polynucleotide, and the additional polynucleotides differ by a Hamming distance of at least 2.

In some embodiments, at least 1% of the first polynucleotide, the second polynucleotide, the third polynucleotide, the fourth polynucleotide, and the additional polynucleotides differ by a Hamming distance of at least 3. In some embodiments, at least 5% of the first polynucleotide, the second polynucleotide, the third polynucleotide, the fourth polynucleotide, and the additional polynucleotides differ by a Hamming distance of at least 3. In some embodiments, at least 10% of the first polynucleotide, the second polynucleotide, the third polynucleotide, the fourth polynucleotide, and the additional polynucleotides differ by a Hamming distance of at least 3. In some embodiments, at least 20% of the first polynucleotide, the second polynucleotide, the third polynucleotide, the fourth polynucleotide, and the additional polynucleotides differ by a Hamming distance of at least 3. In some embodiments, at least 30% of the first polynucleotide, the second polynucleotide, the third polynucleotide, the fourth polynucleotide, and the additional polynucleotides differ by a Hamming distance of at least 3. In some embodiments, at least 40% of the first polynucleotide, the second polynucleotide, the third polynucleotide, the fourth polynucleotide, and the additional polynucleotides differ by a Hamming distance of at least 3. In some embodiments, at least 45% of the first polynucleotide, the second polynucleotide, the third polynucleotide, the fourth polynucleotide, and the additional polynucleotides differ by a Hamming distance of at least 3. In some embodiments, at least 50% of the first polynucleotide, the second polynucleotide, the third polynucleotide, the fourth polynucleotide, and the additional polynucleotides differ by a Hamming distance of at least 3.

In some embodiments, at least 10% of the first polynucleotide, the second polynucleotide, the third polynucleotide, the fourth polynucleotide, the additional polynucleotides, and the synonymous polynucleotides differ by a Hamming distance of at least 2. In some embodiments, at least 25% of the first polynucleotide, the second polynucleotide, the third polynucleotide, the fourth polynucleotide, the additional polynucleotides, and the synonymous polynucleotides differ by a Hamming distance of at least 2. In some embodiments, at least 50% of the first polynucleotide, the second polynucleotide, the third polynucleotide, the fourth polynucleotide, the additional polynucleotides, and the synonymous polynucleotides differ by a Hamming distance of at least 2. In some embodiments, at least 90% of the first polynucleotide, the second polynucleotide, the third polynucleotide, the fourth polynucleotide, the additional polynucleotides, and the synonymous polynucleotides differ by a Hamming distance of at least 2. In some embodiments, at least 95% of the first polynucleotide, the second polynucleotide, the third polynucleotide, the fourth polynucleotide, the additional polynucleotides, and the synonymous polynucleotides differ by a Hamming distance of at least 2. In some embodiments, at 100% of the first polynucleotide, the second polynucleotide, the third polynucleotide, the fourth polynucleotide, the additional polynucleotides, and the synonymous polynucleotides differ by a Hamming distance of at least 2.

### Restriction Sites

In some embodiments, polynucleotides described herein (e.g., the first polynucleotide, the second polynucleotide, the third polynucleotide, the fourth polynucleotide, the additional polynucleotide and/or the synonymous polynucleotides) are engineered to comprise restriction enzyme nucleotide motifs (i.e., restriction sites). In some embodiments, restriction sites are introduced into a polynucleotide by introducing one or more non-synonymous or synonymous codons into the polynucleotide such that a restriction site is formed. In some embodiments, the restriction enzyme is engineered into the nucleic acids encoding the gene variant. In some embodiments, restriction sites are introduced into a polynucleotide by introducing one or more non-synonymous or synonymous codons into the polynucleotide. In some embodiments, introducing the restriction sites does not change the amino acid sequence of the variant gene product encoded by the polynucleotide. Restriction sites are well known in the art, and include, but are not limited to the restriction sites listed in Table 1.

| Enzyme | Recognition Sequence |
|---|---|
| EcoRI | 5′GAATTC |
| | 3′CTTAAG |
| EcoRII | 5′CCWGG |
| | 3′GGWCC |
| BamHI | 5′GGATCC |
| | 3′CCTAGG |
| HindIII | 5′AAGCTT |
| | 3′TTCGAA |
| TaqI | 5′TCGA |
| | 3′AGCT |
| NotI | 5′GCGGCCGC |
| | 3′CGCCGGCG |
| HinFI | 5′GANTC |
| | 3′CTNAG |
| Sau3AI | 5′GATC |
| | 3′CTAG |
| PvuII* | 5′CAGCTG |
| | 3′GTCGAC |
| SmaI* | 5′CCCGGG |
| | 3′GGGCCC |
| HaeIII* | 5′GGCC |
| | 3′CCGG |
| HgaI | 5′GACGC |
| | 3′CTGCG |
| AluI* | 5′AGCT |
| | 3′TCGA |
| EcoRV* | 5′GATATC |
| | 3′CTATAG |
| EcoP15I | 5′CAGCAGN25NN |
| | 3′GTCGTCN25NN |
| KpnI | 5′GGTACC |
| | 3′CCATGG |
| PstI | 5′CTGCAG |
| | 3′GACGTC |
| SacI | 5′GAGCTC |
| | 3′CTCGAG |
| SalI | 5′GTCGAC |
| | 3′CAGCTG |
| ScaI | 5′AGTACT |
| | 3′TCATGA |
| SpeI | 5′ACTAGT |
| | 3′TGATCA |
| SphI | 5′GCATGC |
| | 3′CGTACG |
| StuI | 5′AGGCCT |
| | 3′TCCGGA |
| XbaI | 5′TCTAGA |
| | 3′AGATCT |

W = A or T
N = C or G or T or A
* = blunt ends

In some embodiments, a polynucleotide is engineered to comprise two restriction sites that create complementary overhang regions. In some embodiments, the two restriction sites allow for a portion of the polynucleotide to be removed by restriction digest and subsequent ligation.

In some embodiments, the polynucleotide is engineered to comprise a restriction site to allow for an insertion into a given polynucleotide by performing a restriction digestion and subsequent ligation of the insertion.

### Plasmids, Vectors and Cells

In some embodiments, this disclosure provides a plurality of plasmids comprising polynucleotides of the plurality of polynucleotides. The plurality of plasmids may be produced by ligating the plurality of polynucleotides into a plurality of plasmids with an empty backbone and isolating plasmids with successful ligation of the polynucleotide (e.g., using standard molecular biology techniques). In some embodiments, the plurality of plasmids collectively comprises at least 80% (e.g., at least 90%, at least 95%, or at least 99%) of the different gene variants in the plurality of polynucleotides. In some embodiments, the plurality of plasmids collectively comprises 100% of the different gene variants in the plurality of polynucleotides.

In some embodiments, this disclosure provides a plurality of vectors comprising polynucleotides of the plurality of polynucleotides. In some embodiments, the vector is a viral vector (e.g., an adeno-associated vector, a lentiviral vector, or an adenoviral vector). In some embodiments, the plurality of vectors collectively comprises at least 80% (e.g., at least 90%, at least 95%, or at least 99%) of the different gene variants in the plurality of polynucleotides. In some embodiments, the plurality of vectors collectively comprises 100% of the different gene variants in the plurality of polynucleotides.

In some embodiments, this disclosure provides a plurality of cells collectively comprising polynucleotides of the plurality of polynucleotides, plasmids of the plurality of plasmids or vectors of the plurality of vectors. In some embodiments, a plurality of cells that collectively comprises a plurality of polynucleotides refers to a plurality of cells where at least some of the cells of the plurality of cells (e.g. most of the cells or all of the cells) each comprise a polynucleotide of the plurality of polynucleotides such that each polynucleotide of the plurality of polynucleotides is found in at least one cell of the plurality of cells. This similarly applies to a plurality of cells collectively comprising plasmids of the plurality of plasmids and/or vectors of the plurality of vectors. In some embodiments, the cells of the plurality of cells are bacterial cells (e.g., E. coli strains DH5α, DH10B, BL21, BL21-DE3). In some embodiments, the plurality of cells are mammalian, yeast or insect cells. In some embodiments, the plurality of cells are human cells. In some embodiments, the plurality of cells collectively comprises at least 80% (e.g., at least 90%, at least 95%, or at least 99%) of the different gene variants in the plurality of polynucleotides. In some embodiments, the plurality of cells collectively comprises 100% of the different gene variants in the plurality of polynucleotides.

### Producing Polynucleotides

In some embodiments, this disclosure provides a method of producing a plurality of polynucleotides, the method comprising synthesizing the polynucleotides of a plurality of polynucleotides described herein. In some embodiments, the method comprising synthesizing the first polynucleotide and the second polynucleotide. In some embodiments, the method comprising synthesizing the first polynucleotide, the second polynucleotide, and the third polynucleotide. In some embodiments, the method comprising synthesizing the first polynucleotide, the second polynucleotide, the third polynucleotide, and the fourth polynucleotide. In some embodiments, the method comprising synthesizing the first polynucleotide, the second polynucleotide, the third polynucleotide, the fourth polynucleotide and at least one of the additional polynucleotides. In some embodiments, the method comprising synthesizing the first polynucleotide, the second polynucleotide, the third polynucleotide, the fourth polynucleotide, at least one of the additional polynucleotides, and at least one of the synonymous polynucleotides. In some embodiments, the method comprising synthesizing at least one of the synonymous polynucleotides. In some embodiments, the method comprises synthesizing the plurality of polynucleotides.

Polynucleotides of the plurality of polynucleotides can be synthesized using any suitable method. For example, a plurality of polynucleotides encoding a reference gene may be synthesized by modifying nucleic acids encoding a corresponding reference gene to comprise the synonymous and/or non-synonymous codons, as described herein, using molecular biology (e.g., whole plasmid site-directed mutagenesis, whole-plasmid site-directed mutagenesis with template/product modification, site-directed mutagenesis by overlap extension, inverse PCR, combinatorial codon mutagenesis based, OMNICHANGE as described in Alejaldre et al., BioEssays 43.8 (2021): 2100052. In some embodiments, a polynucleotide may be synthesized using a method comprising silicon-based synthesis or phosphoramidite synthesis (e.g., as described in Hoose, A. Nat Rev Chem 7, 144-161 (2023)). In some embodiments, a polynucleotide may be synthesized enzymatically (e.g. using terminal transferase TdT or a polymerase with terminal transferase activity). Depending on the length of the polynucleotide, the method may comprise synthesizing portions of a given polynucleotide then ligating those portions together or assembling those using Polymerase Cycling Assembly or Overlap Extension PCR (e.g., as described in Hoose, A. Nat Rev Chem 7, 144-161 (2023)).

### Methods of Library Screening

One reason for producing a library described herein is to screen nucleic acid modifying enzyme variant gene products for improved properties (e.g., improved stability, salt tolerability, heat tolerability, etc.). This disclosure provides methods of determining the relative activity of a plurality of nucleic acid modifying enzyme variants of the same gene.

A “nucleic acid modifying enzyme” refers to an enzyme that modifies the primary structure of a polynucleotide. In some embodiments, the nucleic acid modifying enzyme comprises a DNA polymerase. In some embodiments, the DNA polymerase is a Family A Polymerase. In some embodiments, the DNA polymerase is a Family B Polymerase. In some embodiments, the DNA polymerase is a Taq polymerase or a KOD polymerase.

In some embodiments, nucleic acid modifying enzyme activity refers to a measurement of activity of the enzyme. For example, a DNA polymerase activity is to copy DNA, so the number of copies made by the enzyme over a given period of time is one measure of the enzyme's activity. In another example, activity may relate to the fidelity of the enzyme, so for DNA Polymerase a fidelity-base activity may be the number of errors in copies of DNA made by the enzyme. Activity may be a combination of attributes, e.g., a combination of rate of catalysis and fidelity. Activity may be measured in numerous different environments (e.g., high salt and low salt) and different enzymes may have different activities in different environments.

In some embodiments, the method for determining the relative activity of a plurality of nucleic acid modifying enzyme variants of the same gene, comprises one or more of following steps:
obtaining a plurality of cells, wherein at least two of the cells has have been transformed with a polynucleotide of the plurality of polynucleotides described herein;
depositing, into different partitions, (i) individual cells of the plurality of cells, and (ii) reagents sufficient for an active nucleic acid modifying enzyme variant to produce an identifying nucleic acid product that can be used to identify the active nucleic acid modifying enzyme variant in the partition;
lysing the individual cells in the different partitions to combine (i) nucleic acid modifying enzyme variants produced by the cell in the partition and (ii) the reagents sufficient for an active nucleic acid modifying enzyme variant to produce an identifying nucleic acid product;
performing sequencing to determine the amount of the identifying nucleic acid product in at least one of the different partitions; and
determining the relative activity of a plurality of nucleic acid modifying enzyme variants by comparing the amount of at least one identifying nucleic acid product to an amount of identifying nucleic acid products produced in a partition comprising a control nucleic acid modifying enzyme variant.

### Obtaining

In some embodiments, a method comprises obtaining the plurality of cells, wherein at least one of the cells has been transformed with a polynucleotide of the plurality of polynucleotides described herein. In some embodiments, obtaining comprises obtaining cells from any suitable source. In some embodiments, at least two of the cells have been transformed with a polynucleotide of the plurality of polynucleotides described herein. In some embodiments, the obtaining comprises synthesizing the plurality of polynucleotides. In some embodiments, the obtaining comprises introducing a polynucleotide of the plurality of polynucleotides into a cell of the plurality of cells. This may be performed by placing polynucleotides of the plurality of polynucleotides into plasmids or vectors (e.g., such that there is one polynucleotide per plasmid or vector) and transforming the plasmid or transfecting the vector into cells of the plurality of cells. In some embodiments, transforming is performed with a target of less than or equal to 1 plasmid per cells of the plurality of cells. In some embodiments, obtaining the plurality of cells comprises obtaining the plurality of polynucleotides and transforming or transfecting them into a plurality of cells. In some embodiments, the cells are bacterial cells (e.g., E. coli cells designed for cloning or protein production). In some embodiments, the cells selected for production of the specific enzyme in the screen, e.g., a eukaryotic protein may be expressed from a eukaryotic cell.

In some embodiments, a method comprises obtaining a plurality of cells, wherein the plurality of cells collectively comprises at least 25% of the gene variants in the plurality of polynucleotides. In some embodiments, the method comprises obtaining a plurality of cells, wherein the plurality of cells collectively comprises at least 50% of the gene variants in the plurality of polynucleotides. In some embodiments, the method comprises obtaining a plurality of cells, wherein the plurality of cells collectively comprises at least 75% of the gene variants in the plurality of polynucleotides. In some embodiments, the method comprises obtaining a plurality of cells, wherein the plurality of cells collectively comprises at least 90% of the gene variants in the plurality of polynucleotides. In some embodiments, the method comprises obtaining a plurality of cells, wherein the plurality of cells collectively comprises at least 95% of the gene variants in the plurality of polynucleotides. In some embodiments, the method comprises obtaining a plurality of cells, wherein the plurality of cells collectively comprises at least 98% of the gene variants in the plurality of polynucleotides. In some embodiments, the method comprises obtaining a plurality of cells, wherein the plurality of cells collectively comprises at least 99% of the gene variants in the plurality of polynucleotides. In some embodiments, the method comprises obtaining a plurality of cells, wherein the plurality of cells collectively comprises 100% of the gene variants in the plurality of polynucleotides.

### Depositing

In some embodiments, a method comprises depositing, into different partitions, (i) individual cells of the plurality of cells, and (ii) reagents sufficient for an active nucleic acid modifying enzyme variant to produce an identifying nucleic acid product that can be used to identify the active nucleic acid modifying enzyme variant in the partition. “Partitions” refers to separate physical locations. In some embodiments, a partition is a microfluidic droplet (e.g., a water in oil emulsion). In some embodiments, a partition is a vial. In some embodiments, a partition is a well of a plate. In some embodiments, a partition is a spot on a slide or flow cell. In some embodiments, a partition is a bead or microparticle. In some embodiments, a partition is created by physically separating the cells (e.g., using a petri dish). Depositing into different partitions refers to separating the cells into different partitions. However, in some embodiments, this separating may not be perfect. For example, a number of partitions may end up with two cells instead of one. In some embodiments, the different cells of the plurality of cells are different partitions.

In some embodiments, partitions may have additional properties to help identify enzyme variants with specific properties. For example, the partitions may comprise high salt concentrations to identify enzyme variants that are active in high salt. In some embodiments, the partitions may be at a temperature that is above the optimal temperature of the reference enzyme (e.g., wild-type enzyme corresponding to the enzyme variants being tested) to identify enzyme variants that are thermostable. In some embodiments, partitions comprise modified nucleotides in order to identify variants with altered ability to incorporate modified nucleotides into a polynucleotide (e.g., a polymerase incorporating inosine into a DNA polynucleotide). In some embodiments, partitions comprise modified DNA in order to identify variants with altered ability to use modified DNA as template. Similarly, the pH, magnesium concentration, organic solvent percentage, or numerous properties may be modified to identify enzyme variants that perform well in the conditions induced by these properties.

The “reagents sufficient for an active nucleic acid modifying enzyme variant to produce an identifying nucleic acid product” differ based on the type of nucleic acid modifying enzyme variant being screened. For example, for a DNA polymerase the reagents are a DNA template, a primer that is complementary to the template, dNTPs, magnesium ions, and an aqueous buffered solution of a suitable pH. For polymerase (e.g., Taq polymerase or KOD polymerase), the reagents comprise 20 mM Tris-HCl, 10 mM (NH4)2SO4, 10 mM KCl, 2 mM MgSO4, 0.1% Triton® X-100, at pH 8.8.

The “identifying nucleic acid product” refers to a nucleic acid product produced by the enzyme variant in the partition and that is indicative of the enzyme variant in the partition. In some embodiments, the enzyme variant makes copies of its own DNA or RNA. These copies are identifying nucleic acid products because the enzyme that made the copies can be determined by their sequence. Additionally, the amount of the identifying nucleic acid product in the partition is indicative of the activity of the enzyme variant. In some embodiments, the “identifying nucleic acid product” comprises a barcode, e.g., as described herein.

### Lysing

In some embodiments, a method comprises lysing the individual cells in the different partitions to combine (i) nucleic acid modifying enzyme variants produced by the cell in the partition and (ii) the reagents sufficient for an active nucleic acid modifying enzyme variant to produce an identifying nucleic acid product.

Lysing the individual cells may be performed by any suitable means, including but not limited to, heat lysis, mechanical lysis, and chemical lysis (e.g., using non-denaturing detergent or surfactant). Non-denaturing detergents are known in the art and include Triton-X100, NP-40, Tween 20, Tween 40 and CHAPS. Lysing is designed to release the nucleic acid modifying enzyme variant from the cell so that it can interact with the reagent and produce the identifying nucleic acid product. Different lysing methods may be used based on the properties of the nucleic acid modifying enzyme. For example, if the nucleic acid modifying protein is a heat stable polymerase, then lysing by heat would be an acceptable method, whereas lysing by heat may not be acceptable when the nucleic acid modifying protein is not heat stable.

### Performing Sequencing

In some embodiments, a method comprises performing sequencing to determine the amount of the identifying nucleic acid product in at least one of the different partitions. Sequencing may be performed using any suitable method. In some embodiments, performing sequencing comprises performing next-generation sequencing. In some embodiments, performing sequencing comprises performing shotgun sequencing. In some embodiments, the method comprises performing amplicon sequencing. In some embodiments, the method comprises performing short-read sequencing. In some embodiments, the method comprises performing long read sequencing. In some embodiments, performing sequencing comprises performing ILLUMINA sequencing, SOLID sequencing, PACBIO sequencing, or nanopore sequencing. In some embodiments, performing sequencing comprises performing Illumina sequencing. In some embodiments, the method comprises performing PACBIO or nanopore sequencing. In some embodiments, the method comprises performing Sanger sequencing.

In some embodiments, sequencing comprises sequencing the region of the identifying nucleic acid product that comprises the non-synonymous and synonymous codons described herein (including any additional synonymous codons).

In some embodiments, a method comprises combining the different partitions after the identifying nucleic acid product is produced but before sequencing. Some types of sequencing (e.g., Illumina short-read sequencing), is limited in polynucleotide length that can be sequenced (e.g., Illumina sequences up to about 500 nucleotides, but preferably around 250 or less nucleotides, so paired end sequence can be used). However, a total polynucleotide length (specifically the nucleic acids encoding the enzyme variant) may be far longer than 500 nucleotides. In some embodiments that comprise sequencing with Illumina short-read sequencing, the synonymous codons and non-synonymous codons on a given polynucleotide are within 500 nucleotides of one another. In some embodiments that comprise sequencing with Illumina short-read sequencing, the synonymous codons and non-synonymous codons on a given polynucleotide are within 200 nucleotides of one another. In some embodiments that comprise sequencing with short-read sequencing (e.g., Illumina), the synonymous codons and non-synonymous codons on a given polynucleotide are within 100 nucleotides of one another. In some embodiments that comprise sequencing with Illumina short-read sequencing, the synonymous codons and non-synonymous codons on a given polynucleotide are within 30 nucleotides of one another. In contrast, long read methods (e.g., PACBIO and nanopore) can sequence very long polynucleotides.

### Determining Relative Activity

In some embodiments, a method comprises determining the relative activity of a plurality of nucleic acid modifying enzyme variants by comparing the amount of at least one identifying nucleic acid product to an amount of identifying nucleic acid products produced in a partition comprising a control nucleic acid modifying enzyme variant.

The amount of the at least one identifying nucleic acid molecule is determined using data produced by sequencing. This sequencing data includes a measure of the number of sequence reads associated with a given sequence. More sequence reads of a given polynucleotide are indicative of more of the polynucleotide in the original sample (e.g., a partition). Thus, sequencing can identify the sequence of the identifying nucleic acid product (which identifies the enzyme variant), and the activity of the enzyme variant in the partition (based on the number of sequencing reads (i.e., the amount) assigned to the identifying nucleic acid product). The number of sequencing reads may be determined using different methods that are known in the art. For example, in some methods, each identifying nucleic acid product is given a unique molecule identifier (UMI) after production but before sequencing because sample preparation for sequencing (which often include polymerase chain reaction) can skew quantitative measure. A UMI is a polynucleotide barcode. The UMI allows the user to count the number of UMIs associated with a given identifying nucleic acid product, rather than the number of sequencing reads covering the identifying nucleic acid product, in order to more accurately quantify the number of identifying nucleic acid product in a partition prior to any bias introduced by sample preparation.

“Relative activity” refers to the measured activity of a given enzyme variant relative to a control enzyme variant. Relative activity may be calculated by determining a change in activity between the activity of the given enzyme and the activity of the control enzyme. Relative activity may be determined from sequencing data as described below.

$$\frac{( \frac{post}{pre ( var )} )}{μ ( \frac{post}{pre ( wtsyn )} )} \\ \frac{( \frac{post ( var )}{pre ( var )} )}{( \frac{post}{pre ( control )} )} \\ relative activity ( var ) = \frac{\frac{{freq}_{post} ( var )}{{freq}_{pre} ( var )}}{\frac{{freq}_{post} ( control )}{{freq}_{pre} ( control )}}$$

Where relative activity (var) is the calculated activity of an enzyme variant compared to a control enzyme variant, generally a wild-type-like enzyme; freqpost (var) is the count of observations of that variant, divided by the total number of observations at that site (freq), following the screening process (post); freqpre (var) is the frequency of that same variant prior to screening (pre); (freqpost (control) is the frequency of a reference enzyme following the screening process and freqpre (control) prior to screening.

In some embodiments, the control enzyme may be the wild-type enzyme that corresponds to the given enzyme. In some embodiments, the control enzyme variant may be encoded by one or more different polynucleotide sequence variants that encode the same amino acid sequence (e.g., the wild-type enzyme protein sequence). For example, the control enzyme variant may be encoded by a plurality of polynucleotides having different synonymous codons, but encoding the control enzyme variant amino acid sequence. In some embodiments, the plurality of polynucleotides having different synonymous codons can be used to make a plurality of different measurements (one for each polynucleotide variant) of the control enzyme. In some embodiments, the control enzyme variant may be an enzyme variant with known activity. In some embodiments, the control enzyme may actually be a catalytically inactive enzyme.

The “control nucleic acid modifying enzyme variant” is a nucleic acid modifying enzyme selected as a reference point for comparing the activity of different enzyme variants. In some embodiments, the control nucleic acid modifying enzyme variant comprises the nucleic acid sequence of the reference sequence. In some embodiments, the control nucleic acid modifying enzyme variant is a wild-type enzyme corresponding to the enzyme variants. In some embodiments, the control nucleic acid modifying enzyme variant is an enzyme variant of known activity and/or utility. For example, in an assay to determine activity of the enzyme variants at elevated temperature, an enzyme with known elevated temperature activity may be used as a control.

In some embodiments, after lysing, the temperature in the different partitions may be altered. For example, when the polynucleotide modifying enzyme is a DNA polymerase, the heating and cooling cycles of the polymerase chain reaction may be applied to the partitions to promote production of the identifying nucleic acid product (e.g., by amplification of the DNA encoding the DNA polymerase variant by the DNA polymerase variant).

### EXAMPLES

### Example 1. Supercodons Increase the Accuracy of High Throughput Compartmentalized Self-Replication (CSR) Screens of Thermus aquaticus (Taq) Polymerase Variants

Compartmentalized self-replication (CSR) is a directed evolution technique developed to screen the activity of many enzyme variants in a high throughput fashion using next-generation sequencing. Supercodons are three consecutive mutated codons that result in a synonymous codon, a non-synonymous codon, and a synonymous codon. Enzyme variants that deviate from wild-type enzymes in their performance typically have nonsynonymous codons, or mutations in coding regions that result in an amino acid that differ from the amino acid present in the wild-type enzyme. Synonymous codons are mutations in coding regions that result in the same amino acid as the reference sequence (e.g., wild-type enzyme amino acid sequence) and are expected to not alter enzymatic function. The synonymous mutations of the supercodon increase the Hamming distance between different polynucleotides encoding different enzyme variants, which in turn results in greatly improved accuracy in high throughput CSR.

CSR involves the physical separation of an enzyme variant-expressing library of host cells through water-in-oil emulsions. The resulting droplets serve as picoliter-sized reaction vessels in which reaction reagents like dNTPs and primers can be co-encapsulated. These thermostable emulsions are subjected to thermal cycling, during which the host cells release the expressed enzyme into the droplet, along with the encoding plasmid. Primers that flank the coding sequence of the enzyme on the vector are supplied. During the CSR process, selective pressures can be applied, including higher temperatures or salt concentration to select for thermostability or salt tolerance. The resulting yield of any variant sequence is directly correlated with the activity of the variant in question under the applied selection pressures.

Traditionally, CSR is performed on wild-type enzymes in an attempt to generate high-performing variants, or on variants resulting from random mutagenesis of a wild-type enzyme. Wild-type enzymes and variants with low activity tend to be highly prevalent following a single round of CSR, meaning many rounds of CSR are required for enrichment to identify alternative variants that are high performing (or low performing, depending on the goal of the directed evolution). Additionally, in traditional CSR, following multiple rounds of iterative selection, Sanger sequencing of individual enriched clones can determine sequence-function relationships. However, Sanger sequencing only sequences single DNA fragments at a time, making the process prohibitively slow when screening a high number of variants.

Presented here are libraries of polynucleotides encoding enzyme variants that are subjected to directed evolution and identified via next-generation sequencing. The libraries represent a significant improvement in identifying enzyme variants in a high-throughput, high-coverage manner.

### Methods

To develop a high-coverage variant library of Thermus aquaticus (Taq) polymerase variants, variants were generated by DNA synthesis to cover 19 amino acid substitutions at every position over the entire length of the enzyme. Briefly, the criteria for design of the variants were:
(i) across a 9 bp window centered on the primary codon, maximize the edit distance of the variant sequence from the WT sequence as well as other variant sequences at that position where possible, edit 2 or 3 codons for the amino acid substitution;
(ii) where multiple sequences result in the same total edit distance, choose the supercodon with the smallest change in codon usage frequency—for example, if a rare WT glycine codon (GGA, 0.13%) is being substituted by an arginine, we prefer the codon with most similar frequency (CGG, 0.11%, Δ=−2%); and
(iii) remove codons that result in the generation of an undesirable restriction site.

This library design resulted in the generation of about 15,000 enzyme variants representing 19 nonsynonymous supercodons and 1 synonymous supercodon at every possible residue across the length of the Taq polymerase.

Mutations that arise during the process of CSR, preparation for shotgun sequencing, and sequencing errors introduce noise due to spontaneous mutations or nucleic acid copy errors, making it extremely difficult to determine the sequences of enzyme variants with high accuracy. To address this, synonymous codons were introduced at positions adjacent to the nonsynonymous codons, further differentiating variant sequences from the wild-type enzyme sequence, as well as from other variants with nonsynonymous codons at the same site, by increasing the Hamming distance (edit distance). Mutations enriched for or underrepresented in Taq polymerase variants were therefore identified with high confidence following next generation sequencing after CSR.

### Results

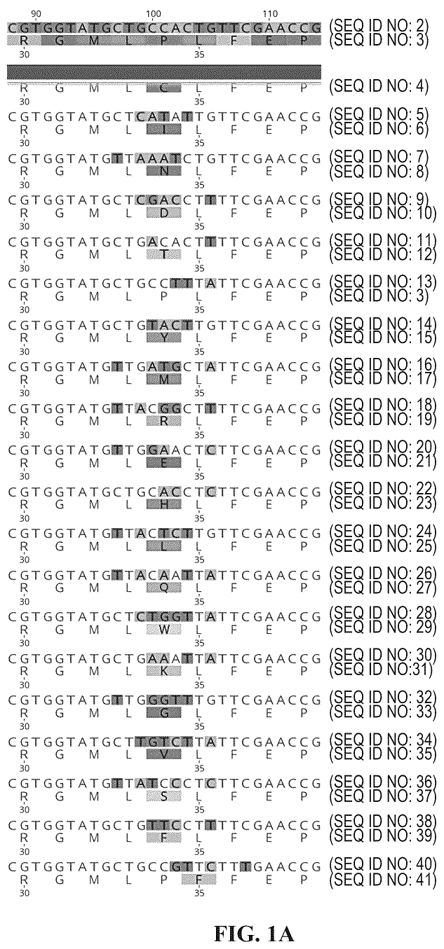

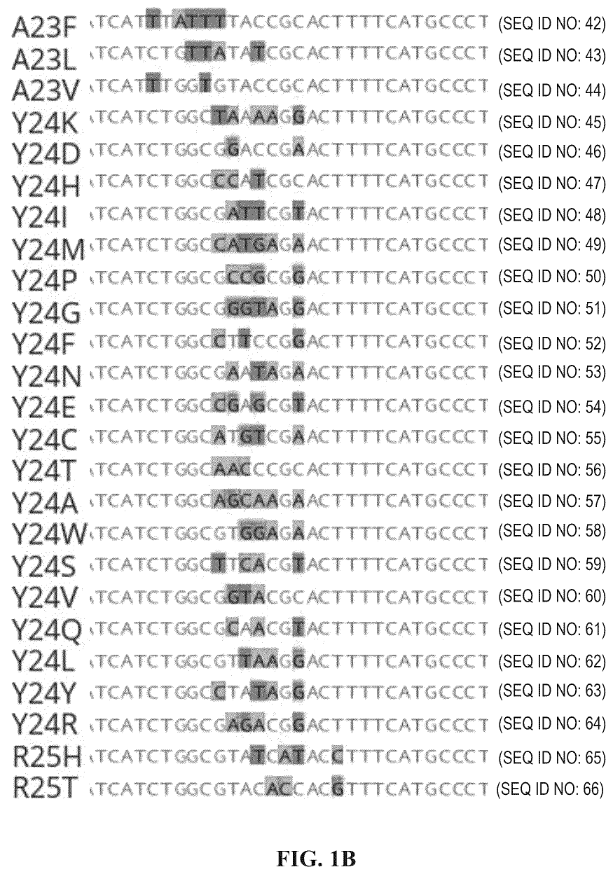

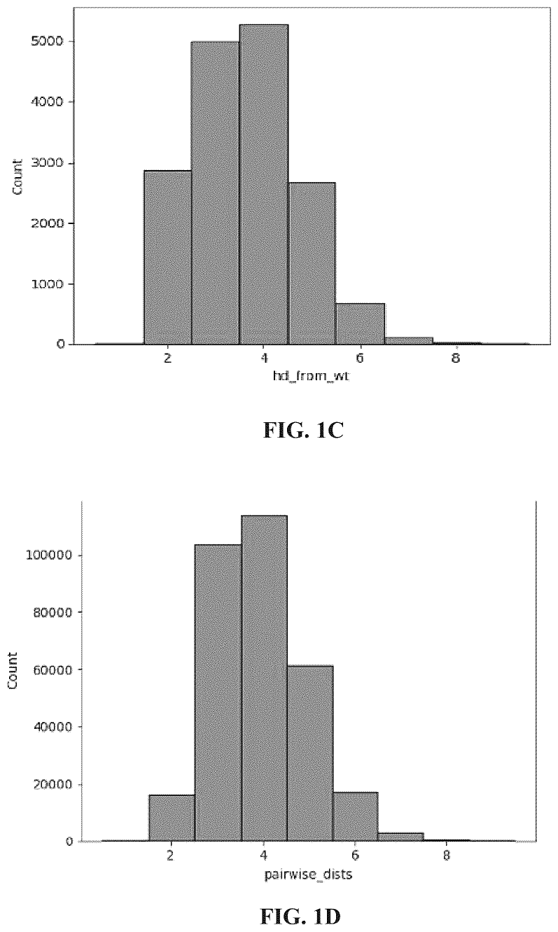

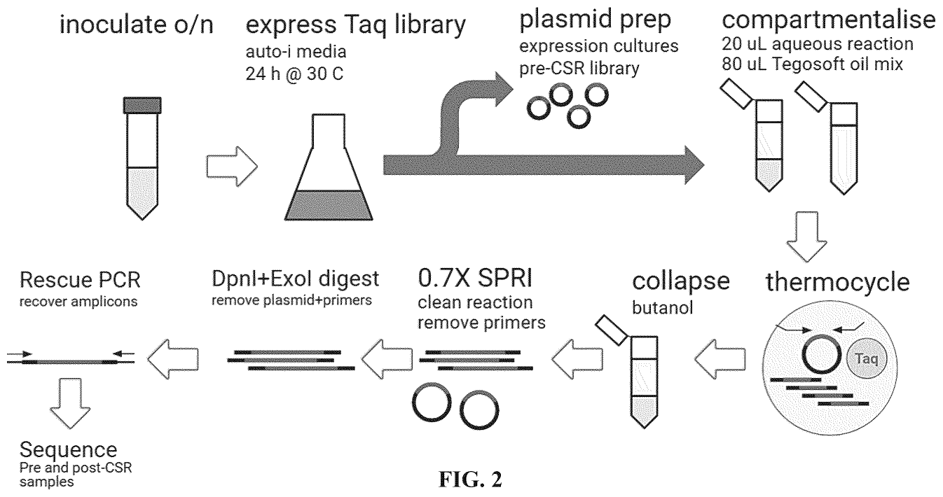

A library of Taq polymerase variants was designed as described above (see FIGS. 1A-1B for example mutations; FIG. 1C shows the distribution of Hamming distances between variants and the reference sequence). FIG. 1D shows the frequencies of pairwise Hamming distances between each member of the library and every other member of the library. Escherichia coli (E. coli) cells were transformed with plasmids carrying the Taq polymerase variants and subjected to CSR in a buffer containing 40 mM Tris-Cl pH 8.3, 40 mM KCl, 200 uM each dNTP, 2 mM MgCl2 and primers flanking the Taq gene library sequences. Following CSR, rescue polymerase chain reaction (PCR), with the Equinox Amplification Mastermix, and next-generation sequencing, on an Illumina Novaseq instrument, was performed (FIG. 2).

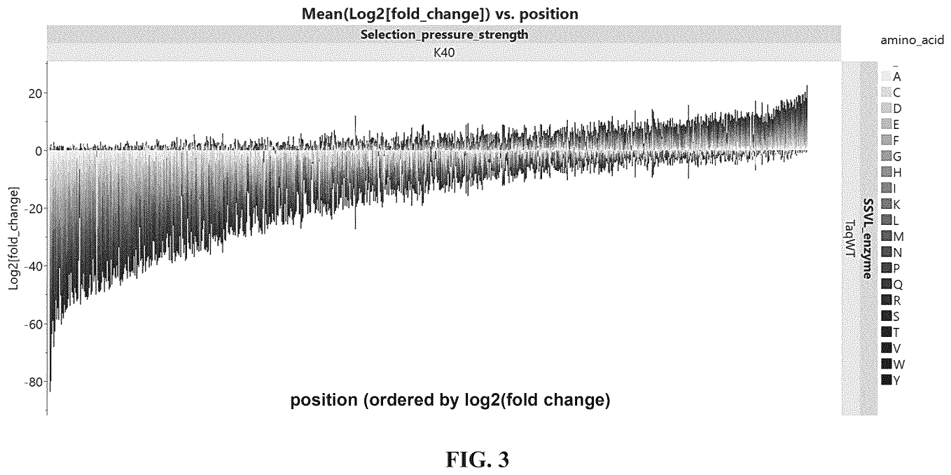

Based on the sequencing data, the enrichment of amino acid substitutions at each position of the Taq polymerase was determined, resulting in a near-complete readout of the sequence-function relationship of all single substitutions (FIG. 3). In FIG. 3, each gene variant is presented by a spike on the graph. Increasing positive values indicate an increase in the relative activity of the gene product. Decreasing negative values indicate a decrease in the relative activity of the gene product.

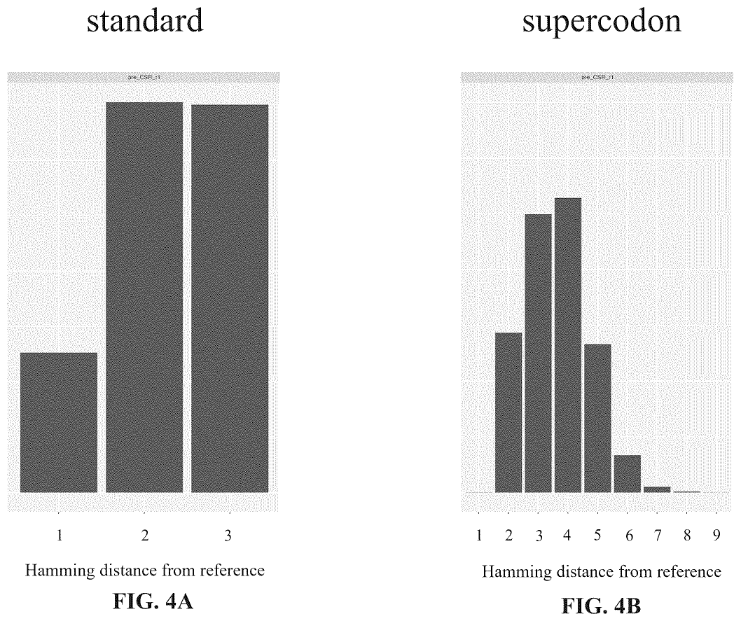

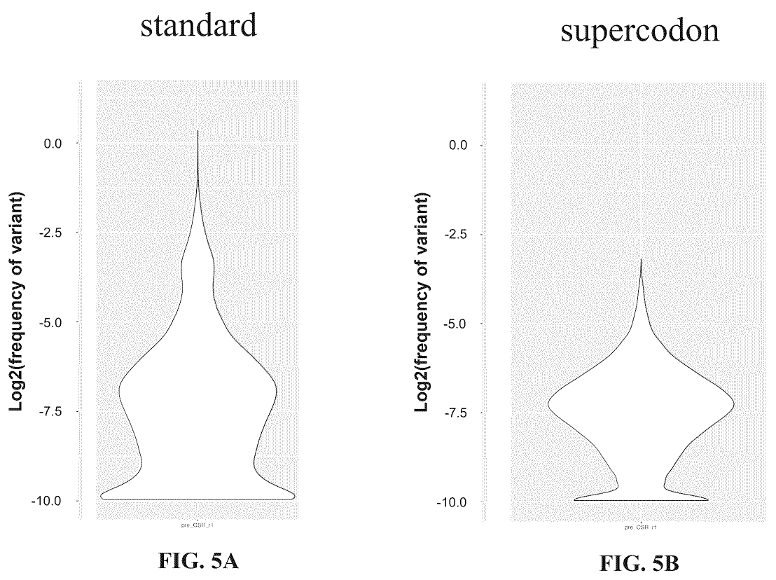

These data demonstrate that enzyme variant libraries as described herein allow for high-throughput, full-coverage directed evolution of enzymes using CSR, and that sequence-function relationships can be identified with high accuracy using next-generation sequencing. The sensitivity of the determination of variant frequencies in this experiment is improved by the incorporation of synonymous substitutions (e.g. supercodons) in the library design (FIG. 4). FIG. 5A shows the artificially inflated variant frequencies resulting from a standard library design (without supercodons). The majority of this inflation is contributed by variants with Hamming distances of one from the reference sequence, which make up a significant proportion of the library (FIG. 4A). In contrast, variant frequencies show a much tighter distribution and dramatically reduced inflation when a supercodon library design is used (FIG. 5B). This is enabled by the almost complete avoidance of Hamming distance one variants (FIG. 4B).

### ADDITIONAL EMBODIMENTS

Additional embodiments provided by the disclosure are set forth in the numbered paragraphs below.

1. A library comprising a plurality of polynucleotides that encode different variants of the same gene product, wherein the plurality comprises: (i) a first polynucleotide comprising nucleic acids encoding a first variant, the nucleic acids comprising a first non-synonymous codon at a first position and a synonymous codon (e.g., a first synonymous codon); and (ii) a second polynucleotide comprising nucleic acids encoding a second variant, the nucleic acids comprising a second non-synonymous codon at the first position and a synonymous codon (e.g., a second synonymous codon).
2. The library of paragraph 1, wherein the plurality of polynucleotides comprises a third polynucleotide, wherein the third polynucleotide comprises nucleic acids encoding a third variant, the nucleic acids comprising: (i) a non-synonymous codon at the first position that is different from the first non-synonymous codon and the second non-synonymous codon; and (ii) a synonymous codon (e.g., a third synonymous codon).
3. The library of paragraph 2, wherein the plurality of polynucleotides comprises a fourth polynucleotide, wherein the fourth polynucleotide comprises: nucleic acids encoding a fourth variant, the nucleic acids comprising; (i) a non-synonymous codon at a different position than the first position; and (ii) a synonymous codon (e.g., the fourth synonymous codon).
4. The library of any one of paragraphs 1-3, wherein the first position is within 200 nucleotides of the synonymous codon.
5. The library of any one of paragraphs 4-3, wherein the first position is within 30 nucleotides of the synonymous codon.
6. The library of paragraph 1, wherein the first position is within 3 nucleotides of the synonymous codon.
7. The library of any one of paragraphs 3-6, wherein the nucleic acids of the first polynucleotide, the nucleic acids of the second polynucleotide, the nucleic acids of the third polynucleotide, and/or the nucleic acids of the fourth polynucleotide further comprise an additional synonymous codon.
8. The library of any one of paragraphs 1-7, wherein the first polynucleotide comprises, in consecutive codons: a synonymous codon; the first non-synonymous codon; and an additional synonymous codon.
9. The library of any one of paragraphs 1-8, wherein the second polynucleotide comprises in consecutive codons: a synonymous codon; the second non-synonymous codon; and an additional synonymous codon.
10. The library of any one of paragraphs 2-9, wherein the third polynucleotide comprises, in consecutive codons: a synonymous codon; the non-synonymous codon at the first position that is different from the first non-synonymous codon and the second non-synonymous codon; and an additional synonymous codon.
11. The library of any one of paragraphs 3-7, wherein the fourth polynucleotide comprises, in consecutive codons: a synonymous codon; the non-synonymous codon at a different position than the first position; and an additional synonymous codon.
12. The library of any one of paragraphs 1-11, wherein the first polynucleotide and the second polynucleotide differ by a Hamming distance of at least 2.
13. The library of any one of paragraphs 1-12, wherein: the first non-synonymous codon encodes a specific amino acid and is selected to have a codon usage percentage that is closest to the codon usage percentage of a codon at the first position of a polynucleotide that encodes a reference sequence of the gene product; the second non-synonymous codon encodes a specific amino acid and is selected to have a codon usage percentage that is closest to a codon usage percentage of a codon at the second position of a polynucleotide that encodes a reference sequence of the gene product; the third non-synonymous codon encodes a specific amino acid and is selected to have a codon usage percentage that is closest to a codon usage percentage of a codon at the third position of a polynucleotide that encodes a reference sequence of the gene product; and/or the fourth non-synonymous codon encodes a specific amino acid and is selected to have a codon usage percentage that is closest to a codon usage percentage of a codon at the fourth position of a polynucleotide that encodes a reference sequence of the gene product.
14. The library of paragraph 13, wherein the reference sequence encodes the wild-type amino acid sequence of the gene product.
15. The library of paragraph 13, wherein the reference sequence comprises the wild-type nucleic acid sequence of the gene product.
16. The library of any one of paragraphs 1-12, wherein: the first non-synonymous codon has a codon usage percentage that is within 10% of the codon usage percentage of a codon at the first position of a polynucleotide that encodes a reference sequence of the gene product; the second non-synonymous codon has a codon usage percentage that is within 10% of the codon usage percentage of a codon at the second position of a polynucleotide that encodes a reference sequence of the gene product; the third non-synonymous codon has a codon usage percentage that is within 10% of the codon usage percentage of a codon at the third position of a polynucleotide that encodes a reference sequence of the gene product; and/or the fourth non-synonymous codon has a codon usage percentage that is within 10% of the codon usage percentage of a codon at fourth position of a polynucleotide that encodes a reference sequence of the gene product.
17. The library of any one of paragraphs 1-16, further comprising additional polynucleotides that each comprise nucleic acids encoding different variants of the gene product, wherein the nucleic acids of each of the additional polynucleotides comprise: (i) a non-synonymous codon; and (ii) a synonymous codon.
18. The library of paragraphs 17, wherein the non-synonymous codon is within 200 nucleotides of the synonymous codon.
19. The library of paragraphs 17, wherein the non-synonymous codon is within 30 nucleotides of the synonymous codon.
20. The library of paragraph 17, wherein the non-synonymous codon is within 3 nucleotides of the synonymous codon.
21. The library any one of paragraphs 17-20, wherein the nucleic acids of at least one of the additional polynucleotides further comprises an additional synonymous codon.
22. The library of paragraph 21, wherein at least one of the additional polynucleotides comprises, in consecutive codons: a synonymous codon; the non-synonymous codon; and an additional synonymous codon.
23. The library of any one of paragraphs 17-22, wherein 1%-100% of the first polynucleotide, the second polynucleotide, the third polynucleotide, the fourth polynucleotide, and the additional polynucleotides differ by a Hamming distance of at least 2.
24. The library of any one of paragraphs 17-22, wherein the first polynucleotide, the second polynucleotide, the third polynucleotide, and the additional polynucleotides collectively comprise nucleic acids encoding at least 75% of possible amino acid substitutions at the first position.
25. The library of any one of paragraphs 17, wherein the first polynucleotide, the second polynucleotide, the third polynucleotide, and the additional polynucleotides collectively comprise nucleic acids encoding at least 90% of possible amino acid substitutions at the first position.
26. The library of any one of paragraphs 25, wherein the first polynucleotide, the second polynucleotide, the third polynucleotide, and the additional polynucleotides collectively comprise nucleic acids encoding 100% of possible amino acid substitutions at the first position.
27. The library of any one of paragraphs 3-26, wherein the first polynucleotide, the second polynucleotide, the third polynucleotide, the fourth polynucleotide, and the additional polynucleotides collectively comprise nucleic acids encoding at least 10% of possible single amino acid substitutions of the gene product.
28. The library of any one of paragraph 27, wherein the first polynucleotide, the second polynucleotide, the third polynucleotide, the fourth polynucleotide, and the additional polynucleotides collectively comprise nucleic acids encoding at least 50% of possible single amino acid substitutions of the gene product.
29. The library of paragraph 27, wherein the first polynucleotide, the second polynucleotide, the third polynucleotide, the fourth polynucleotide, and the additional polynucleotides collectively comprise nucleic acids encoding at least 75% of possible single amino acid substitutions of the gene product.
30. The library of paragraph 27, wherein the first polynucleotide, the second polynucleotide, the third polynucleotide, the fourth polynucleotide, and the additional polynucleotides collectively comprise nucleic acids encoding at least 90% of possible single amino acid substitutions of the gene product.
31. The library of paragraph 27, wherein the first polynucleotide, the second polynucleotide, the third polynucleotide, the fourth polynucleotide, and the additional polynucleotides collectively comprise nucleic acids encoding at least 95% of possible single amino acid substitutions of the gene product.
32. The library of paragraph 27, wherein the first polynucleotide, the second polynucleotide, the third polynucleotide, the fourth polynucleotide, and the additional polynucleotides collectively comprise nucleic acids encoding at least 99% of possible single amino acid substitutions of the gene product.
33. The library of any one of paragraphs 1-32, wherein the gene product comprises a nucleic acid modifying enzyme.
34. The library of paragraph 33, wherein the nucleic acid modifying enzyme is a DNA polymerase.
35. The library of paragraph 33, wherein the nucleic acid modifying enzyme is a Family A polymerase.
36. The library of paragraph 33, wherein the nucleic acid modifying enzyme is a Family B polymerase.
37. The library of paragraph 34, wherein the DNA polymerase is a Thermococcus kodakarensis (KOD) DNA polymerase or a Taq polymerase.
38. The library of any one of paragraphs 3-34, wherein the first polynucleotide, the second polynucleotide, the third polynucleotide, the fourth polynucleotide, and the additional polynucleotides collectively comprise nucleic acids encoding at least 25% of the possible single non-synonymous codons in the gene product.
39. The library of paragraph 38, wherein the first polynucleotide, the second polynucleotide, the third polynucleotide, the fourth polynucleotide, and the additional polynucleotides collectively comprise nucleic acids encoding at least 50% of the possible single non-synonymous codons of the gene product.
40. The library of paragraph 38, wherein the first polynucleotide, the second polynucleotide, the third polynucleotide, the fourth polynucleotide, and the additional polynucleotides collectively comprise nucleic acids encoding at least 75% of the possible single non-synonymous codons of the gene product.
41. The library of any one of paragraphs 38, wherein the first polynucleotide, the second polynucleotide, the third polynucleotide, the fourth polynucleotide, and the additional polynucleotides collectively comprise nucleic acids encoding at least 95% of the possible single non-synonymous codon of the gene product.
42. The library of any one of paragraphs 1-41, wherein the plurality of polynucleotides comprises a polynucleotide comprising nucleic acids encoding a wild-type of the gene product.
43. The library of any one of paragraphs 1-42, wherein the first polynucleotide, the second polynucleotide, the third polynucleotide, the fourth polynucleotide, and the additional polynucleotides collectively comprise nucleic acids encoding at least 100 different variants of the same gene product.
44. The library of any one of paragraphs 1-43, wherein the first polynucleotide, the second polynucleotide, the third polynucleotide, the fourth polynucleotide, and the additional polynucleotides collectively comprise nucleic acids encoding at least 500 different variants of the same gene product.
45. The library of any one of paragraphs 1-44, wherein the first polynucleotide, the second polynucleotide, the third polynucleotide, the fourth polynucleotide, and the additional polynucleotides collectively comprise nucleic acids encoding at least 5,000 different variants of the same gene product.
46. The library of any one of paragraphs 1-45, wherein the first polynucleotide, the second polynucleotide, the third polynucleotide, the fourth polynucleotide, and the additional polynucleotides collectively comprise nucleic acids encoding at least 10,000 different variants of the same gene product.
47. The library of any one of paragraphs 1-46, wherein the first polynucleotide, the second polynucleotide, the third polynucleotide, the fourth polynucleotide, and the additional polynucleotides collectively comprise nucleic acids encoding at least 20,000 different variants of the same gene product.
48. The library of any one of paragraphs 1-47, wherein the first polynucleotide, the second polynucleotide, the third polynucleotide, the fourth polynucleotide, and the additional polynucleotides collectively comprise nucleic acids encoding at least 30,000 different variants of the same gene product.
49. The library of any one of paragraphs 1-48, wherein each polynucleotide encodes a single gene variant of the same gene product.
50. The library of any one of paragraphs 1-48, wherein the nucleic acids of the first polynucleotide, the nucleic acids of the second polynucleotide, the nucleic acids of the third polynucleotide, the nucleic acids of the fourth polynucleotide, and/or the nucleic acids of an additional polynucleotide comprise a synonymous codon or non-synonymous codon that introduces a restriction site.
51. The library of any one of paragraphs 1-48, wherein the nucleic acids of the first polynucleotide, the nucleic acids of the second polynucleotide, the nucleic acids of the third polynucleotide, the nucleic acids of the fourth polynucleotide, and/or the nucleic acids of an additional polynucleotide comprise a synonymous codon that introduces a restriction site.
52. A library comprising a plurality of polynucleotides that encode different variants of the same gene product, wherein the plurality comprises: (i) a first polynucleotide comprising nucleic acids encoding a first variant, wherein the first polynucleotide comprises a first non-synonymous codon and a synonymous codon; and (ii) a second polynucleotide comprising nucleic acids encoding a second variant, wherein the second polynucleotide comprises a second non-synonymous codon and a synonymous codon.
53. The library of any one of paragraphs 1-52, wherein the plurality comprises a synonymous polynucleotide comprising nucleic acids of a gene variant encoding the gene product, wherein the synonymous polynucleotide comprises a first synonymous codon and a second synonymous codon.
54. A library comprising a plurality of polynucleotides that encode different variants of the same gene, wherein the plurality comprises a synonymous polynucleotide comprising nucleic acids of a gene variant encoding the gene product, wherein the synonymous polynucleotide comprises a first synonymous codon and a second synonymous codon.
55. The library of paragraph 54, wherein the first synonymous codon is within 200 nucleotides of the second synonymous codon on the first polynucleotide.
56. The library of paragraph 54, wherein the first synonymous codon is within 30 nucleotides of the second synonymous codon on the first polynucleotide.
57. The library of paragraph 54, wherein the first synonymous codon is within 3 nucleotides of the second synonymous codon on the first polynucleotide.
58. The library of any one of paragraphs 53-57, wherein the synonymous polynucleotide comprises a third synonymous codon.
59. The library of paragraph 58, wherein the synonymous polynucleotide comprises, in consecutive codons: the first synonymous codon; the second synonymous codon; and the third synonymous codon.
60. A library comprising the plurality of polynucleotides of any one of paragraphs 1-51, the plurality of polynucleotides of paragraph 53, and the plurality of polynucleotides of any one of paragraphs 54-59.
61. The library of any one of paragraphs 1-60, wherein a polynucleotide of the plurality of polynucleotides comprises a barcode.
62. A plurality of plasmids that comprise a polynucleotide of the library of any one of paragraphs 1-61.
63. A plurality of vectors that encode a polynucleotide of the library of any one of paragraphs 1-61.
64. A plurality of cells that comprise:
(i) a polynucleotide of the library of any one of paragraphs 1-61;
(ii) a plasmid of the plurality of plasmids of paragraphs 62; and/or
(iii) a vector of the plurality of vectors of paragraph 63.
   65. A method of producing a plurality of polynucleotides, the method comprising synthesizing the first polynucleotide and the second polynucleotide of any one of paragraphs 1-61.
   66. The method of paragraph B1, further comprising synthesizing the third polynucleotide of any one of paragraphs 2-61.
   67. The method of paragraph B1 or paragraph B2, further comprising synthesizing the fourth polynucleotide of any one of paragraphs 3-61.
   68. The method of any one of paragraphs 65-67, further comprising synthesizing at least one of the additional polynucleotides of any one of paragraphs 17-61.
   69. The method of any one of paragraphs 65-68, wherein the nucleic acids of the first polynucleotide, the nucleic acids of the second polynucleotide, the nucleic acids of the third polynucleotide, the nucleic acids of the fourth polynucleotide, the nucleic acids of the at least one of the additional polynucleotides, and/or the nucleic acids of the synonymous polynucleotide further comprise an additional nonsynonymous codon.
   70. A method for determining the relative activity of a plurality of nucleic acid modifying enzyme variants of the same gene, the method comprising: obtaining a plurality of cells, wherein at least one of the cells has been transformed with a polynucleotide of the plurality of polynucleotides of any one of paragraphs 1-61; depositing, into different partitions, (i) individual cells of the plurality of cells, and (ii) reagents sufficient for an active nucleic acid modifying enzyme variant to produce an identifying nucleic acid product that can be used to identify the active nucleic acid modifying enzyme variant in the partition; lysing the individual cells in the different partitions to combine (a) nucleic acid modifying enzyme variants produced by the cell in the partition and (b) the reagents sufficient for an active nucleic acid modifying enzyme variant to produce an identifying nucleic acid product; performing sequencing to determine the amount of the identifying nucleic acid product in at least one of the different partitions; and determining the relative activity of a plurality of nucleic acid modifying enzyme variants by comparing the amount of at least one identifying nucleic acid product to an amount of identifying nucleic acid products produced in a partition comprising a control nucleic acid modifying enzyme variant.
   71. The method of paragraph 70, wherein at least 2 cells of the plurality of cells have been transformed with a polynucleotide of the plurality of polynucleotides of any one of paragraphs 1-61.
   72. The method of paragraph 70, wherein each cell of the plurality of cells has been transformed with a polynucleotide of the plurality of polynucleotides of any one of paragraphs 1-61.
   73. The method of paragraph 70, wherein the control nucleic acid modifying enzyme variant comprises a wild-type nucleic acid modifying enzyme of the same gene as the nucleic acid modifying enzyme variants.
   74. The method of any one of paragraphs 70-72, wherein the control nucleic acid modifying enzyme variant comprises a nucleic acid modifying variant of known function.
   75. The method of paragraph 73, wherein the control nucleic acid modifying enzyme variant comprises a synonymous codon.
   76. The method of any one of paragraphs 70-74, wherein the different partitions are different wells in a plate or different vials.
   77. The method of any one of paragraphs 70-76, wherein the different partitions are water-in-oil emulsions.
   78. The method of any one of paragraphs 70-77, wherein the lysing the individual cells comprises lysing the individual cells with at least one surfactant and/or lysing individuals cells using heat.
   79. The method of any one of paragraphs 70-78, wherein determining the relative activity of a nucleic acid modifying enzyme variant comprises determining an enrichment of an identifying nucleic acid product corresponding to the nucleic acid modifying enzyme variant relative to the control nucleic acid modifying enzyme variant.
   80. The method of any one of paragraphs 70-79, wherein performing sequencing comprises performing shotgun sequencing.
   81. The method of any one of paragraphs 70-79, wherein performing sequencing comprises performing long-read sequencing.

1. A method for determining the relative activity of a plurality of nucleic acid modifying enzyme variants of the same gene product, the method comprising:
(i) obtaining a plurality of cells that collectively comprise a plurality of polynucleotides, wherein polynucleotides of the plurality of polynucleotides encode different nucleic acid modifying enzyme variants of the same gene product, wherein the plurality of polynucleotides comprises:
(a) a first polynucleotide comprising nucleic acids encoding a first variant, the nucleic acids comprising a first non-synonymous codon at a first position and a first synonymous codon; and
(b) a second polynucleotide comprising nucleic acids encoding a second variant, the nucleic acids comprising a second non-synonymous codon at the first position and a second synonymous codon,
wherein the first non-synonymous codon is different than the second non-synonymous codon;
(ii) depositing, into different partitions, (a) individual cells of the plurality of cells, and (b) reagents sufficient for an active nucleic acid modifying enzyme variant to produce an identifying nucleic acid product that can be used to identify the active nucleic acid modifying enzyme variant in the partition;
(iii) lysing the individual cells in the different partitions to combine (a) a nucleic acid modifying enzyme variant produced by the cell in the partition and (b) the reagents sufficient for an active nucleic acid modifying enzyme variant to produce an identifying nucleic acid product;
(iv) performing sequencing to determine an amount of the identifying nucleic acid products of the first variant and an amount of the identifying nucleic acid products of the second variant; and
(v) determining: (a) the relative activity of the first variant by comparing the amount of the identifying nucleic acid products of the first variant to an amount of an identifying nucleic acid product produced in a partition comprising a control nucleic acid modifying enzyme variant; and (b) the relative activity of the first variant by comparing the amount of the identifying nucleic acid products of the second variant to an amount of an identifying nucleic acid product produced in a partition comprising a control nucleic acid modifying enzyme variant.

2. A method for determining the relative activity of a plurality of nucleic acid modifying enzyme variants of the same gene product, the method comprising:
(i) obtaining a plurality of cells, wherein at least one of the cells has been transformed with a polynucleotide of a plurality of polynucleotides encoding a plurality of nucleic acid modifying enzyme variants of the same gene, wherein the plurality of polynucleotides comprises:
(a) a first polynucleotide comprising nucleic acids encoding a first variant, the nucleic acids comprising a first non-synonymous codon at a first position and a first synonymous codon; and
(b) a second polynucleotide comprising nucleic acids encoding a second variant, the nucleic acids comprising a second non-synonymous codon at the first position and a second synonymous codon, wherein the first non-synonymous codon is different than the second non-synonymous codon;
(ii) depositing, into different partitions, (a) individual cells of the plurality of cells, and (a) reagents sufficient for an active nucleic acid modifying enzyme variant to produce an identifying nucleic acid product that can be used to identify the active nucleic acid modifying enzyme variant in the partition;
(iii) lysing the individual cells in the different partitions to combine (a) a nucleic acid modifying enzyme variant produced by the cell in the partition and (b) the reagents sufficient for an active nucleic acid modifying enzyme variant to produce an identifying nucleic acid product;
(iv) performing sequencing to determine the amount of the identifying nucleic acid product in at least one of the different partitions; and
(v) determining the relative activity of a plurality of nucleic acid modifying enzyme variants by comparing the amount of at least one identifying nucleic acid product to an amount of identifying nucleic acid products produced in a partition comprising a control nucleic acid modifying enzyme variant.

3. The method of claim 1, wherein:
the first polynucleotide comprises, in consecutive codons: the first synonymous codon; the first non-synonymous codon; and a first additional synonymous codon; and
the second polynucleotide comprises, in consecutive order, the second synonymous codon; the second non-synonymous codon; and a second additional synonymous codon.

4. The method of claim 1, wherein the plurality of polynucleotides comprises a third polynucleotide, wherein the third polynucleotide comprises nucleic acids encoding a third variant, the nucleic acids comprising:
(i) a third non-synonymous codon at the first position that is different from the first non-synonymous codon and the second non-synonymous codon; and
(ii) a third synonymous codon.

5. The method of claim 4, wherein the plurality of polynucleotides comprises a fourth polynucleotide, wherein the fourth polynucleotide comprises: nucleic acids encoding a fourth variant, the nucleic acids comprising:
(i) a fourth non-synonymous codon at a different position than the first position; and
(ii) a fourth synonymous codon.

6. The method of claim 5, wherein the plurality of polynucleotides further comprises additional polynucleotides that each comprise nucleic acids encoding different variants of the nucleic acid modifying enzyme, wherein the nucleic acids of each of the additional polynucleotides comprise:
(i) a non-synonymous codon; and
(ii) a synonymous codon.

7. The method of claim 6, wherein performing sequencing comprises performing sequencing to determine an amount of the identifying nucleic acid products of the third variant, an amount of the identifying nucleic acid products of the fourth variant, and/or an amount of the identifying nucleic acid products of one or more additional variants.

8. The method of claim 7, further comprising determining the relative activity of the third variant, the fourth variant and/or one or more of the additional variants.

9. The method of claim 5, wherein the Hamming distance is at least 2 between the nucleic acids encoding the first variant, the nucleic acids encoding the second variant, the nucleic acids encoding the third variant, and the nucleic acids encoding the fourth variant.

10. The method of claim 7, wherein the first polynucleotide, the second polynucleotide, the third polynucleotide, the fourth polynucleotide, and the additional polynucleotides collectively comprise nucleic acids encoding at least 50% of possible single amino acid substitutions of the gene product.

11. The method of claim 10, wherein the first polynucleotide, the second polynucleotide, the third polynucleotide, the fourth polynucleotide, and the additional polynucleotides collectively comprise nucleic acids encoding at least 90% of possible single amino acid substitutions of the gene product.

12. The method of claim 10, wherein the Hamming distance is at least 2 among at least 90% of the nucleic acids encoding the nucleic acid modifying enzyme variants of the polynucleotides of the plurality of polynucleotides.

13. The method of claim 1, wherein the plurality of polynucleotides comprises a set of polynucleotides encoding at least 15 different amino acid substitutions at the first position, wherein each polynucleotide of the set of polynucleotides comprises a non-synonymous codon at the first position and a synonymous codon.

14. (canceled)

15. The method of claim 13, wherein the Hamming distance is at least 2 among the polynucleotides of the set of polynucleotides.

16. The method of claim 6, wherein:
the first non-synonymous codon and the first synonymous codon are within 30 nucleotides of one another;
the second non-synonymous codon and the second synonymous codon are within 30 nucleotides of one another;
the third non-synonymous codon and the third synonymous codon are within 30 nucleotides of one another;
the fourth non-synonymous codon and the fourth synonymous codon are within 30 nucleotides of one another; and
for each additional polynucleotide, the non-synonymous codon and the synonymous codon are within 30 nucleotides of one another.

17. The method of claim 2, wherein at least 2 cells of the plurality of cells have been transformed with a polynucleotide of the plurality of polynucleotides.

18.-27. (canceled)

28. The method of claim 1, wherein the nucleic acid modifying enzyme variants of the same gene product are polymerase variants of the same polymerase.

29. (canceled)

30. A library comprising a plurality of polynucleotides that encode different variants of the same gene product, wherein the plurality comprises:
(i) a first polynucleotide comprising nucleic acids encoding a first variant, the nucleic acids comprising a first non-synonymous codon at a first position and a first synonymous codon; and
(ii) a second polynucleotide comprising nucleic acids encoding a second variant, the nucleic acids comprising a second non-synonymous codon at the first position and a second synonymous codon.

31.-36. (canceled)

37. The library of claim 30, wherein the first polynucleotide comprises, in consecutive codons: the first synonymous codon; the first non-synonymous codon; and an additional synonymous codon.

38.-82. (canceled)

83. A library comprising a plurality of polynucleotides that encode different variants of the same gene, wherein the plurality comprises a synonymous polynucleotide comprising nucleic acids of a gene variant encoding the gene product, wherein the synonymous polynucleotide comprises a first synonymous codon and a second synonymous codon.

84-93. (canceled)

94. A method of producing a plurality of polynucleotides, the method comprising synthesizing the first polynucleotide and the second polynucleotide of claim 30.

95.-98. (canceled)


SEQUENCE LISTING (66 sequences)

SEQ ID NO: 1 - RNA, 12 nt, synthetic construct
  source 1..12: mol_type = other RNA
tttttaataatg

SEQ ID NO: 2 - DNA, 27 bp, synthetic construct
  source 1..27: mol_type = other DNA
cgtggtatgctgccactgttcgaaccg

SEQ ID NO: 3 - protein, 9 aa, synthetic construct
RGMLPLFEP

SEQ ID NO: 4 - protein, 9 aa, synthetic construct
RGMLCLFEP

SEQ ID NO: 5 - DNA, 27 bp, synthetic construct
  source 1..27: mol_type = other DNA
cgtggtatgctcatattgttcgaaccg

SEQ ID NO: 6 - protein, 9 aa, synthetic construct
RGMLILFEP

SEQ ID NO: 7 - DNA, 27 bp, synthetic construct
  source 1..27: mol_type = other DNA
cgtggtatgttaaatctgttcgaaccg

SEQ ID NO: 8 - protein, 9 aa, synthetic construct
RGMLNLFEP

SEQ ID NO: 9 - DNA, 27 bp, synthetic construct
  source 1..27: mol_type = other DNA
cgtggtatgctcgaccttttcgaaccg

SEQ ID NO: 10 - protein, 9 aa, synthetic construct
RGMLDLFEP

SEQ ID NO: 11 - DNA, 27 bp, synthetic construct
  source 1..27: mol_type = other DNA
cgtggtatgctgacacttttcgaaccg

SEQ ID NO: 12 - protein, 9 aa, synthetic construct
RGMLTLFEP

SEQ ID NO: 13 - DNA, 27 bp, synthetic construct
  source 1..27: mol_type = other DNA
cgtggtatgctgcctttattcgaaccg

SEQ ID NO: 14 - DNA, 27 bp, synthetic construct
  source 1..27: mol_type = other DNA
cgtggtatgctgtacttgttcgaaccg

SEQ ID NO: 15 - protein, 9 aa, synthetic construct
RGMLYLFEP

SEQ ID NO: 16 - DNA, 27 bp, synthetic construct
  source 1..27: mol_type = other DNA
cgtggtatgttgatgctattcgaaccg

SEQ ID NO: 17 - protein, 9 aa, synthetic construct
RGMLMLFEP

SEQ ID NO: 18 - DNA, 27 bp, synthetic construct
  source 1..27: mol_type = other DNA
cgtggtatgttacggcttttcgaaccg

SEQ ID NO: 19 - protein, 9 aa, synthetic construct
RGMLRLFEP

SEQ ID NO: 20 - DNA, 27 bp, synthetic construct
  source 1..27: mol_type = other DNA
cgtggtatgttggaactcttcgaaccg

SEQ ID NO: 21 - protein, 9 aa, synthetic construct
RGMLELFEP

SEQ ID NO: 22 - DNA, 27 bp, synthetic construct
  source 1..27: mol_type = other DNA
cgtggtatgctgcacctcttcgaaccg

SEQ ID NO: 23 - protein, 9 aa, synthetic construct
RGMLHLFEP

SEQ ID NO: 24 - DNA, 27 bp, synthetic construct
  source 1..27: mol_type = other DNA
cgtggtatgttactcttgttcgaaccg

SEQ ID NO: 25 - protein, 9 aa, synthetic construct
RGMLLLFEP

SEQ ID NO: 26 - DNA, 27 bp, synthetic construct
  source 1..27: mol_type = other DNA
cgtggtatgttacaattattcgaaccg

SEQ ID NO: 27 - protein, 9 aa, synthetic construct
RGMLQLFEP

SEQ ID NO: 28 - DNA, 27 bp, synthetic construct
  source 1..27: mol_type = other DNA
cgtggtatgctctggttattcgaaccg

SEQ ID NO: 29 - protein, 9 aa, synthetic construct
RGMLWLFEP

SEQ ID NO: 30 - DNA, 27 bp, synthetic construct
  source 1..27: mol_type = other DNA
cgtggtatgctgaaattattcgaaccg

SEQ ID NO: 31 - protein, 9 aa, synthetic construct
RGMLKLFEP

SEQ ID NO: 32 - DNA, 27 bp, synthetic construct
  source 1..27: mol_type = other DNA
cgtggtatgttgggtttgttcgaaccg

SEQ ID NO: 33 - protein, 9 aa, synthetic construct
RGMLGLFEP

SEQ ID NO: 34 - DNA, 27 bp, synthetic construct
  source 1..27: mol_type = other DNA
cgtggtatgcttgtcttattcgaaccg

SEQ ID NO: 35 - protein, 9 aa, synthetic construct
RGMLVLFEP

SEQ ID NO: 36 - DNA, 27 bp, synthetic construct
  source 1..27: mol_type = other DNA
cgtggtatgttatccctcttcgaaccg

SEQ ID NO: 37 - protein, 9 aa, synthetic construct
RGMLSLFEP

SEQ ID NO: 38 - DNA, 27 bp, synthetic construct
  source 1..27: mol_type = other DNA
cgtggtatgctgttccttttcgaaccg

SEQ ID NO: 39 - protein, 9 aa, synthetic construct
RGMLFLFEP

SEQ ID NO: 40 - DNA, 27 bp, synthetic construct
  source 1..27: mol_type = other DNA
cgtggtatgctgccgttctttgaaccg

SEQ ID NO: 41 - protein, 9 aa, synthetic construct
RGMLPFFEP

SEQ ID NO: 42 - DNA, 30 bp, synthetic construct
  source 1..30: mol_type = other DNA
tcatttattttaccgcacttttcatgccct

SEQ ID NO: 43 - DNA, 30 bp, synthetic construct
  source 1..30: mol_type = other DNA
tcatctgttatatcgcacttttcatgccct

SEQ ID NO: 44 - DNA, 30 bp, synthetic construct
  source 1..30: mol_type = other DNA
tcatttggtgtaccgcacttttcatgccct

SEQ ID NO: 45 - DNA, 30 bp, synthetic construct
  source 1..30: mol_type = other DNA
tcatctggctaaaaggacttttcatgccct

SEQ ID NO: 46 - DNA, 30 bp, synthetic construct
  source 1..30: mol_type = other DNA
tcatctggcggaccgaacttttcatgccct

SEQ ID NO: 47 - DNA, 30 bp, synthetic construct
  source 1..30: mol_type = other DNA
tcatctggcccatcgcacttttcatgccct

SEQ ID NO: 48 - DNA, 30 bp, synthetic construct
  source 1..30: mol_type = other DNA
tcatctggcgattcgtacttttcatgccct

SEQ ID NO: 49 - DNA, 30 bp, synthetic construct
  source 1..30: mol_type = other DNA
tcatctggccatgagaacttttcatgccct

SEQ ID NO: 50 - DNA, 30 bp, synthetic construct
  source 1..30: mol_type = other DNA
tcatctggcgccgcggacttttcatgccct

SEQ ID NO: 51 - DNA, 30 bp, synthetic construct
  source 1..30: mol_type = other DNA
tcatctggcgggtaggacttttcatgccct

SEQ ID NO: 52 - DNA, 30 bp, synthetic construct
  source 1..30: mol_type = other DNA
tcatctggccttccggacttttcatgccct

SEQ ID NO: 53 - DNA, 30 bp, synthetic construct
  source 1..30: mol_type = other DNA
tcatctggcgaatagaacttttcatgccct

SEQ ID NO: 54 - DNA, 30 bp, synthetic construct
  source 1..30: mol_type = other DNA
tcatctggccgagcgtacttttcatgccct

SEQ ID NO: 55 - DNA, 30 bp, synthetic construct
  source 1..30: mol_type = other DNA
tcatctggcatgtcgaacttttcatgccct

SEQ ID NO: 56 - DNA, 30 bp, synthetic construct
  source 1..30: mol_type = other DNA
tcatctggcaacccgcacttttcatgccct

SEQ ID NO: 57 - DNA, 30 bp, synthetic construct
  source 1..30: mol_type = other DNA
tcatctggcagcaagaacttttcatgccct

SEQ ID NO: 58 - DNA, 30 bp, synthetic construct
  source 1..30: mol_type = other DNA
tcatctggcgtggagaacttttcatgccct

SEQ ID NO: 59 - DNA, 30 bp, synthetic construct
  source 1..30: mol_type = other DNA
tcatctggcttcacgtacttttcatgccct

SEQ ID NO: 60 - DNA, 30 bp, synthetic construct
  source 1..30: mol_type = other DNA
tcatctggcggtacgcacttttcatgccct

SEQ ID NO: 61 - DNA, 30 bp, synthetic construct
  source 1..30: mol_type = other DNA
tcatctggcgcaacgtacttttcatgccct

SEQ ID NO: 62 - DNA, 30 bp, synthetic construct
  source 1..30: mol_type = other DNA
tcatctggcgttaaggacttttcatgccct

SEQ ID NO: 63 - DNA, 30 bp, synthetic construct
  source 1..30: mol_type = other DNA
tcatctggcctataggacttttcatgccct

SEQ ID NO: 64 - DNA, 30 bp, synthetic construct
  source 1..30: mol_type = other DNA
tcatctggcgagacggacttttcatgccct

SEQ ID NO: 65 - DNA, 30 bp, synthetic construct
  source 1..30: mol_type = other DNA
tcatctggcgtatcatacctttcatgccct

SEQ ID NO: 66 - DNA, 30 bp, synthetic construct
  source 1..30: mol_type = other DNA
tcatctggcgtacaccacgtttcatgccct